(19) United States
(12) Patent Application Publication
(10) Pub. No.: US 2007/0004765 A1
(43) Pub. Date: Jan. 4, 2007

(54) New compounds

(75) Inventors: Malin Graffner-Nordberg, Uppsala (SE); Tjeerd Barf, Ravenstei (NL); Andrew Mott, Knivsta (SE)

Correspondence Address: FISH & RICHARDSON PC, P.O. BOX 1022, MINNEAPOLIS, MN, 55440-1022, US

(73) Assignee: Biovitrum AB, Stockholm (SE)

(21) Appl. No.: 11/437,465

(22) Filed: May 19, 2006

Related U.S. Application Data
(60) Provisional application No. 60/692,339, filed on Jun. 20, 2005.

(30) Foreign Application Priority Data
May 20, 2005 (SE) 0501148-1

(51) Int. Cl. A61K 31/4747; C07D 471/22

(52) U.S. Cl. 514/278; 546/17

(57) ABSTRACT

The present invention relates to compounds of Formula (I):

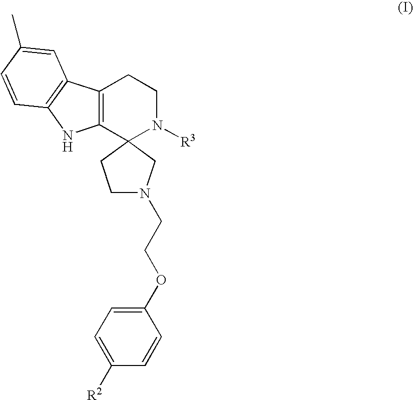

wherein R¹ to R³ are as described herein, processes for preparing the compounds, pharmaceutical compositions comprising the compounds, and use of the compounds and compositions in the prophylaxis or treatment of a GHSR receptor-related disorder. Examples of such disorders are obesity and related disorders such as diabetes type II, dyslipidemia and the metabolic syndrome Prader-Willi syndrome, cardiovascular diseases such as atherosclerotic vascular disease, angina pectoris, myocardial infarction and stroke, intestinal inflammation that is associated with inflammatory bowel diseases (IBD) such as Crohn's disease and ulcerative colitis, acromegaly and cancer, in particular breast, lung, prostate, thyroid and endocrine pituary carcinomas.

### CROSS REFERENCE TO RELATED APPLICATIONS

This application claims the benefit of United States Provisional Application No.: 60/692,339, filed on Jun. 20, 2005 and Swedish Patent Application No.: 0501148-1, filed on May 20, 2005. The contents of these two prior applications are incorporated herein by reference in their entireties.

### TECHNICAL FIELD

The present invention relates to novel compounds, to pharmaceutical compositions that include the compounds, to processes for their preparation, the use of the compounds for the preparation of a medicament against GHSR receptor-related disorders, and methods for the prophylaxis and treatment of GHSR receptor-related disorders.

### BACKGROUND OF THE INVENTION

Ghrelin is a peptide containing 28 amino acids. Kojima and coworkers reported the isolation of Ghrelin from rat stomach extracts in 1999 (Kojima M, et al. (1999) “Ghrelin is a growth-hormone-releasing acylated peptide from stomach.” Nature 402: 656-660). The peptide includes an N-octanoylation of the third residue, Ser³. The presence of this post-translational modification is believed to contribute to the function of the peptide (see, e.g., Kojima et al., supra) and appears to be involved with transport of the peptide across the blood-brain barrier ( see, e.g., Banks W A, et al. (2002) “Extent and direction of ghrelin transport across the blood-brain barrier is determined by its unique primary structure.” J Pharmacol Exp Ther 302:822-827). GHS-R is a G-protein coupled receptor that was cloned in 1996 by Howard and coworkers (Howard A D, et al. (1996) “A receptor in pituitary and hypothalamus that functions in growth hormone release.” Science 273: 974-977). This receptor was found to bind several small synthetic ligands with GH releasing effects known as growth hormone secretagogues (GHS). GHS-R was later identified as a receptor for ghrelin. Des-Gln¹⁴-ghrelin is another endogenous ligand for GHS-R and is believed to form as a result of alternative splicing of the ghrelin gene (see, e.g., Hosoda H, et al. (2000) “Purification and characterization of rat des-Gln14-Ghrelin, a second endogenous ligand for the growth hormone secretagogue receptor.” J Biol Chem 275: 21995-22000).

Ghrelin has been shown to be a potent stimulator of adiposity and food intake in rodents (see, e.g., Tschop M, et al. (2000) “Ghrelin induces adiposity in rodents.” Nature 407:908-913; Wren A M, et al. (2000) “The novel hypothalamic peptide ghrelin stimulates food intake and growth hormone secretion.” Endocrinology 141:4325-4328; Nakazato M, et al. (2001) “A role for ghrelin in the central regulation of feeding.” Nature 409:194-198). Weight gain has been observed following both single daily subcutaneous doses and ICV dosing. The effect has been shown to be present in GHRH deficient rodents pointing at a non-pituitary growth hormone mediated effect (Tschop et al., supra). In contrast, lesions in the hypothalamic arcuate nucleus, believed to be a key area for regulation of energy homeostasis, appeared to essentially eliminate the orexigenic effects but did not appear to affect GH release of exogenously administered ghrelin in rats (see, e.g., Tamura H, et al. (2002) “Ghrelin stimulates GH but not food intake in arcuate nucleus ablated rats.” Endocrinology 143:3268-3275). Antagonists against GHSR have been shown to decrease base-line food intake, weight gain, and energy expenditure suggesting that there is a tonic activation of this receptor that can be down regulated providing further support to the role of GHSR as a target for obesity related disease therapy. Further evidence for ghrelin as a key component in the regulation of the metabolism comes from the tissue distribution of the peptide and receptor. Messenger RNA and protein for ghrelin and GHS-R have been shown to be relatively abundant in many intestinal tissues as well as in several brain tissues, in particular the arcuate nucleus of the hypothalamus (see, e.g., Wang G, et al. (2002) “Ghrelin-not just another stomach hormone.” Regul Pept 105:75-81 for a detailed review). In addition, GHS-R can also be detected in prostate cancers and several other tumours (Jeffery P L, et al. (2003) “The potential autocrine/paracrine roles of ghrelin and its receptor in hormone-dependent cancer” Cytokine Growth Factor Rev 14:113-122).

The release of Ghrelin has been shown to occur in a pulsative manner, characterized by gradual increases before meal and relatively rapid decreases afterwards, suggesting a role in meal initiation and termination in humans (Cummings D E, et al. (2001) “A preprandial rise in plasma ghrelin levels suggests a role in meal initiation in humans.” Diabetes 50:1714-1719). Several studies appear to have established the role of ghrelin in food intake and regulation of body composition in humans (see, e.g., Wren A M, et al. (2001) “Ghrelin enhances appetite and increases food intake in humans.” J Clin Endocrinol Metab 86:5992). Healthy volunteers were reported to eat significantly more after systemic administration of ghrelin, and the effect was still significant 24 hours after the injection.

Apart from the regulatory effects on food intake and metabolism, Ghrelin may also possess anxiogenic and cardiovascular effects. Ghrelin, when injected ICV or peripherally, dose-dependently, has been observed to decrease time spent in the open arm of an elevated plus-maze as well as number of entries (see, e.g., Asakawa A, et al. (2001) A role of ghrelin in neuroendocrine and behavioral responses to stress in mice. Neuroendocrinology 74:143-147). The anxiogenic effect could be blocked by a corticotropin-releasing hormone (CRH) receptor antagonist pointing at an involvement of ghrelin in the hypothalamic-pituitary-adrenal system. Furthermore, chronic administration of ghrelin has also been associated with improved prognosis after heart failure (see, e.g., Nagaya N, et al. (2001) Chronic administration of ghrelin improves left ventricular dysfunction and attenuates development of cardiac cachexia in rats with heart failure. Circulation 104:1430-1435).

Prader-Willi syndrome (PWS) is among the most common forms of human syndromic obesity. It is characterized by severe obesity, hyperphagia, hypogonadism, GH deficiency, neonatal hypotonia, dysmophic features and cognitive impairment. The genetic basis of PWS is believed to involve imprinting disorders of several genes on chromosome 15. PWS patients typically have relatively high fasting-ghrelin concentrations.

Ghrelin has also been shown to be present in pancreatic alpha cells of the rat, where it may act in a paracrine/autocrine fashion to regulate insulin secretion. When administered acutely into human normal young volunteers, ghrelin has been shown to induce hyperglycemia as well as reduce serum levels of insulin.

Finally, it has also been reported that GHSR antagonists could be used against intestinal inflammation that is associated with inflammatory bowel diseases (IBD) such as Crohn's disease and ulcerative colitis (see, e.g., WO 2004/084943, US 2004008385).

### DETAILED DESCRIPTION

This invention relates generally to modulation (e.g., antagonism) of the GHS-R receptor.

In one aspect, this invention relates to a compound of Formula (I):

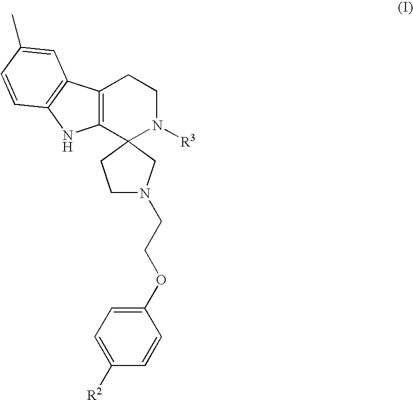

wherein R¹ is a member selected from the group of cyano, SR⁴, SO₂R⁴, N(R⁴)₂, CO₂R⁴, COR⁴, CON(R⁴)₂, O(CO)N(R⁴)₂, OSO₂R⁴, O(CS)N(R⁴)₂, N(R⁴)(CO)N(R⁴)₂, N(R⁴)(CO)R⁴, N(R⁴)(SO₂)R⁴, CH═CHCOOR⁴, CH═CHCON(R⁴)₂, (CH₂)ₙCOOR⁴, (CH₂)ₙCON(R⁴)₂, halo or perhaloalkyl or a heterocycle wherein each R⁴ is independently selected from hydrogen, aryl, heteroaryl, (C₁₋₆)alkyl, (C₂₋₆)alkenyl, (C₂₋₆)alkynyl, (C₂₋₈)heteroalkyl, halo(C₁₋₆)alkyl, or perhalo(C₁₋₆)alkyl; or two R⁴ groups attached to the same nitrogen atom are combined to form a five- to eight-membered heterocyclic ring (in some embodiments, the five- to eight-membered heterocyclic ring can include one or more heteroatoms in addition to the nitrogen atom to which both R⁴ groups are attached; e.g., the five- to eight-membered ring can include an oxygen atom; e.g., two R⁴ groups attached to the same nitrogen atom can combine to form a morpholino ring), and wherein R⁴ is optionally substituted with from one to three substituents independently selected from the group consisting of halogen, hydroxyl, alkoxy, or oxo;

n is 1 to 4;

R² is hydrogen or cyano; and

R³ is selected from hydrogen, an alkyl ester of N-glycylcarbonyl, C₁₋₆-alkyl ester of N-glycylacetyl, carbamoyl-C₁₋₆-alkyl, N-C₁₋₆-alkylcarbamoyl-C₁₋₆-alkyl, N,N-C₁₋₆-dialkylcarbamoyl-C₁₋₆-alkyl, N,N-C₁₋₆-dialkylcarbamoylamino-C₁₋₆-alkyl, C₁₋₆-alkoxy-C₂₋₆-acylamino-C₁₋₆-alkyl, 3-amino-1,2-dioxocyclobut-3-ene-4-ylamino-C₁₋₆-alkyl, 3-C₁₋₆-alkoxy-1,2-dioxocyclobut-3-ene-4-ylamino-C₁₋₆-alkyl, cyano-C₁₋₆-alkyl, C₁₋₆-alkoxyhydroxyalkyl, carboxy-C₁₋₆-alkyl, C₁₋₆-alkoxycarbonyl-C₁₋₆-alkyl, C₁₋₆-alkoxy-C₁₋₆-alkyl, C₁₋₆-alkoxy-C₁₋₆-alkoxy-C₁₋₆-alkyl, aryl-C₁₋₆-alkylamino-C₂₋₆-acyl, C₁₋₆-alkoxycarbonyl-C₁₋₆-alkylamino-C₂₋₆-acyl, carboxy-C₁₋₆-alkylamino-C₂₋₆-acyl, C₂₋₆-acyl-C₂₋₆-acyl, aryloxy-C₁₋₆-alkyl, C₁₋₆-alkylsulfonylamino-C₁₋₆-alkyl, C₁₋₆-alkoxycarbonyl-C₂₋₆-acyl, C₁₋₆-alkoxy-C₂₋₆-acyl, C₁₋₆ -alkylthio-C₂₋₆-acyl, di-C₁₋₆-alkylamino-C₂₋₆-acyl, heteroarylcarbamoyl, C₁₋₆-alkoxycarbonyl, heteroaryl-C₂₋₆-acyl, C₁₋₆-alkylsulfonyl-C₂₋₆-acyl, heterocyclyl-C₂₋₆-acyl, C₁₋₆-alkoxy-C₁₋₆- alkylamino-C₂₋₆-acyl, carboxy-C₂₋₆-acyl, amino-C₂₋₆-acyl, C₁₋₆-alkylamino-C₂₋₆-acyl, carbamoyl-C₁₋₆-alkylamino-C₂₋₆-acyl, heterocyclyl-C₁₋₆-alkyl, heteroaryl-C₁₋₆-alkyl, carbamoylamino-C₁₋₆-alkyl, hydroxy-C₂₋₆-acylcarbamoyl, C₁₋₆-alkylcarbamoyl-C₁₋₆-alkylamino-C₁₋₆-alkyl, C₁₋₆-alkoxycarbonyl-C₁₋₆-alkylamino-C₁₋₆-alkyl, amino-C₂₋₆-acylamino-C₂₋₆-acyl, C₁₋₆-alkoxy-C₂₋₆-acylamino-C₂₋₆-acyl, amino-C₂₋₆-acylamino-C₁₋₆-alkyl, amino-C₂₋₆-acylamino-C₁₋₆-alkyl, heterocyclylcarbonylamino-C₁₋₆-alkyl, C₂₋₆-acylamino-C₁₋₆-alkyl, amino-C₂₋₆-acylamino-C₂₋₆-acyl, C₂₋₆-acylamino-C₂₋₆-acyl, hydroxy-C₁₋₆-alkylamino-C₂₋₆-acyl, C₁₋₆-alkoxycarbonyl-C₁₋₆-alkyl, amino-C₁₋₆-alkyl, carboxy-C₁₋₆-alkyl, 2-(3-hydroxy-1,2-dioxocyclobut-3-ene-4-yl)amino-C₁₋₆-alkyl, heteroarylcarbonylamino-C₁₋₆-alkyl, carboxyamino-C₁₋₆-alkyl, N,N-di-C₁₋₆-alkylamino-C₂₋₆-acylamino-C₁₋₆-alkyl, dihydroxy-C₁₋₆-alkyl, C₂₋₆-acylcarbonyl, C₁₋₆-alkoxybenzyl, wherein the aryl group is optionally substituted by one or more of C₁₋₆-alkoxy, the heteroaryl group is optionally substituted by one or more of C₁₋₆-alkyl and the heterocyclyl is optionally substituted by one or more of oxo.

In some embodiments, R¹ can be trifluoromethyl, 1H-tetrazol-5-yl, cyano, aminocarbonyl, carboxy, morpholin-4-ylcarbonyl, dimethylaminocarbonyl, ethylaminocarbonyl, ethylthio, acetyl, methoxycarbonylethenyl, aminocarbonylethenyl, ethoxycarbonyl, methoxycarbonyl, methylthio, methylsulfonyl, methylsulfonamido, dimethylaminocarbonyloxy, trifluoromethanesulfonyloxy, or dimethylamino(thiocarbonyl)oxy.

In some embodiments, R³ is selected from acetyl, allyl, alkylcarbamoyl, aminoacetyl, 2-(3-amino- 1,2-dioxocyclobut-3-ene-4-ylamino)ethyl, 3-amino-3-methyl-n-butyryl, benzylaminoacetyl, n-butylcarbamoyl, carbamoylmethyl, carbamoylmethylaminoacetyl, 3-carbamoyl-n-propyl, carbethoxy, carbethoxyacetyl, 4-carbethoxy-n-butyl, carbethoxymethyl, 3-carbethoxy-n-propyl, carbomethoxyacetyl, 4-carbomethoxy-n-butyryl, 4-carboxy-n-butyl, 3-carboxy-n-propionyl, 3-carboxy-n-propyl, 3-cyano-n-propyl, cyclohexylcarbamoyl, N,N-diethylcarbamoylmethyl, diisopropylaminoacetyl, 3,4-dimethoxybenzylaminoacetyl, dimethylaminoacetyl, 2-(N,N-dimethylcarbamoylamino)ethyl, 3,5-dimethylisoxazol-4-ylcarbamoyl, 1,4-dioxo-n-pentyl, 2-(3-ethoxy-1,2-dioxocyclobut-3-ene-4-ylamino)ethyl, ethylcarbamoyl, 4-ethylcarbamoyl-n-butyl, 3-ethylcarbamoyl-n-propyl, ethyl ester of N-glycylacetyl, ethyl ester of N-glycylcarbonyl, N-ethyl-N-methylcarbamoyl, ethylthioacetyl, N-glycylacetyl, N-glycylcarbonyl, hydrogen, hydroxyacetyl, 2-hydroxyisobutyl, 2-hydroxyethyl, 2-hydroxy-3-methoxy-n-propyl, 2-hydroxy-n-propyl, 1-imidazolylacetyl, methoxyacetyl, 2-(methoxyacetylamino)ethyl, 2-(2-methoxyethoxy)ethyl, 2-methoxyethylaminoacetyl, 3-methoxy-n-propyl, methyl, methylaminoacetyl, methylsulfonyl, methylsulfonylacetyl, 2-methylsulfonylaminoethyl, 4-morpholinylacetyl, 2-(4-morpholinyl)ethyl, 3-oxo-1-piperazinylacetyl, 2-phenoxyethyl, 1-piperazinylacetyl, 2-pyridylmethyl, 2-thienylcarbamoyl, 2-carbamoylaminoethyl, hydroxyacetylcarbamoyl, 2-(N-methylcarbamoylmethylamino)ethyl, 2-carbomethoxymetylaminoethyl, 2-amino-2-methylpropionamidoacetyl, methoxyacetylaminoacetyl, 2-(2-amino-2-methylpropionamido)ethyl, 2-aminoacetylaminoethyl, 2-(4-morpholinylcarbonylamino)ethyl, 2-acetylaminoethyl, aminoacetylaminoacetyl, acetylaminoacetyl, 2-hydroxyethylaaminoacetyl, carbomethoxymethyl, 2-aminoethyl, carboxymethyl, 2-(3-hydroxy-1,2-dioxocyclobut-3-ene-4-yl)aminoethyl, 2-(2-furylcarbonylamino)ethyl, 2-(5-isoxazolylcarbonylamino)ethyl, 2-carboxyaminoethyl, 2-(2-morpholinylcarbonylamino)ethyl, 2-N,N-dimethylaminoacetylaminoethyl, 4-phenoxy-n-butyl, 2,3-dihydroxy-n-propyl, acetylcarbonyl, and 4-methoxybenzyl.

In some embodiments, R² can be hydrogen (H).

In one aspect, this invention relates to any of the compounds described herein, such as the compounds described in Examples 1-27.

Included in the invention are pharmaceutically acceptable salts, hydrates, solvates, geometrical isomers, tautomers, optical isomers, N-oxides and/or prodrug forms of compounds of the Formula I.

In some embodiments, the compound can have one chiral carbon atom (i.e., the chiral carbon atom shared by the spiro-fused rings in formula (I)). The compound can be a racemic mixture of the (R)-enantiomer and the (S)-emantiomer.

In some embodiments, the compound can be a mixture of the (R)-enantiomer and the (S)-emantiomer that is other than a racemic mixture.

In certain embodiments, the compound can be an enantiomer mixture having at least about 60% (e.g., at least about 70%, at least about 75%, at least about 80%, at least about 85%, at least about 90%, at least about 95%, at least about 99%) of the (R)-enantiomer. The compound can be substantially free of the (S)-emantiomer (i.e., the compound can be the (R)-enantiomer and be substantially free of the (S)-emantiomer). The compound can be the (R)-enantiomer in substantially pure form (i.e., the compound can be the (R)-enantiomer and be substantially free of the (S)-emantiomer as well as other non-stereoisomer-related materials, e.g., solvents, reagents, reaction by-products and the like).

In other embodiments, the compound can be an enantiomer mixture having at least about 60% (e.g., at least about 70%, at least about 75%, at least about 80%, at least about 85%, at least about 90%, at least about 95%, at least about 99%) of the (S)-enantiomer. The compound can be substantially free of the (R)-enantiomer (i.e., the compound can be the (S)-enantiomer and be substantially free of the (R)-enantiomer). The compound can be the (S)-enantiomer in substantially pure form (i.e., the compound can be the (S)-enantiomer and be substantially free of the (R)-emantiomer as well as other non-stereoisomer-related materials, e.g., solvents, reagents, reaction by-products and the like).

In some embodiments, the compound can have two (or more) chiral carbon atoms ((e.g., two chiral carbon atoms, e.g., in which one of the chiral carbon atoms is the carbon atom shared by the spiro-fused rings in formula (I) and the other is a chiral carbon atom that is present in R³). In certain embodiments, R³ can be 2,3-dihydroxy-n-propyl.

In certain embodiments, the compound can be a stereoisomer mixture having at least about 60% (e.g., at least about 70%, at least about 75%, at least about 80%, at least about 85%, at least about 90%, at least about 95%, at least about 99%) of one of the four possible stereoisomers (e.g., R,R; S,S; R,S; or S,R). The compound can be substantially free of its enantiomer and the other two possible stereoisomers. The compound can be one of the four possible stereoisomers in substantially pure form.

In certain embodiments, the compound can be a stereoisomer mixture having at least about 60% (e.g., at least about 70%, at least about 75%, at least about 80%, at least about 85%, at least about 90%, at least about 95%, at least about 99%) of two of the four possible stereoisomers (e.g., R,R and S,S; R,S and S,R; R,R and S,R; R,R and R,S; S,S and S,R; or S,S and S,S and R,S).

In another aspect, this invention relates to a process for the preparation of the compounds described herein. The following synthetic schemes illustrate some of the methods by which the compounds of the invention can be prepared.

In some embodiments, the compounds of formula (I) can be prepared by carrying out a carboxy-mediated Pictet-Spengler reaction (see, e.g., Scheme 1 below). In certain embodiments, a carboxy-mediated Pictet-Spengler reaction can be used to prepare formula (I) compounds that contain electron withdrawing groups (e.g., compounds in which R¹ is an electron withdrawing substituent (e.g., SO₂NH_{2,} NO₂, CH₃CO)).

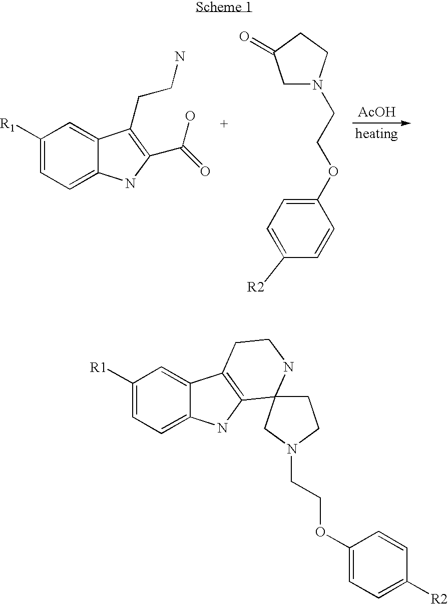

Scheme 2 outlines a reaction scheme in which, for example, 1′-(2-Phenoxyethyl)-2,3,4,9-tetrahydrospiro[beta-carboline-1,3′-pyrrolidin]-6-yl trifluoromethanesulfonate, can be prepared, and the triflate group can then be substituted by a variety of groups (R¹) using palladium catalysed reactions, for example those described in Palladium Reagents and Catalysts by Juro Tsuji, John Wiley and sons (1995). In addition, the palladium-mediated reaction can give rise to an intermediate which can then undergo further chemical transformations (for example cyano is converted to 1H-tetrazol-5-yl).

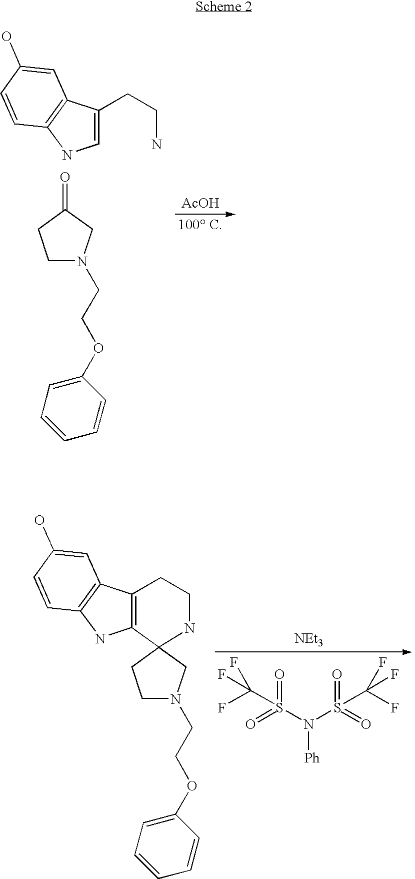

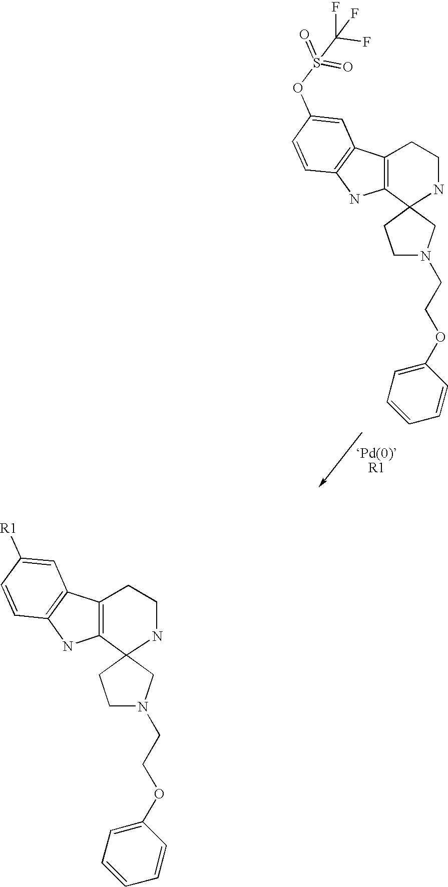

In a further aspect, this invention relates to any compound described herein for use in therapy, e.g., for use in the prophylaxis or treatment of a GHSR receptor-related disorder.

In one aspect, this invention relates to a pharmaceutical formulation that includes one or more of the compounds described here as an active ingredient, in combination with a pharmaceutically acceptable diluent or carrier(e.g., for use in the prophylaxis or treatment of a GHSR receptor-related disorder). In some embodiments, the composition can include an amount of the compound that is effective for the prophylaxis or treatment of a GHSR receptor-related disorder.

In another aspect, this invention relates to a method for treating a human or animal subject suffering from a GHSR receptor-related disorder. The method can include administering to a subject (e.g., a human or an animal, dog, cat, horse, cow) in need thereof an effective amount of one or more compounds of any of the formulae herein, their salts, or compositions containing the compounds or salts.

The methods delineated herein can also include the step of identifying that the subject is in need of treatment of the GHSR receptor-related disorder. Identifying a subject in need of such treatment can be in the judgment of a subject or a health care professional and can be subjective (e.g., opinion) or objective (e.g., measurable by a test or diagnostic method).

In a further aspect, this invention relates to a method for the prophylaxis of a GHSR receptor-related disorder, which can include administering to a subject in need of such treatment an effective amount of one or more of the compounds described herein.

In one aspect, this invention relates to a method for modulating (e g, promoting or inhibiting) GHSR receptor activity, which can include administering to a subject in need of such treatment an effective amount of one or more of the compounds described herein.

In another aspect, this invention relates to a method for suppressing food intake, which can include administering to a subject in need of such treatment an effective amount of one or more of the compounds described herein.

In a further aspect, this invention relates to a method for suppressing appetite, which can include administering to a subject in need of such treatment an effective amount of one or more of the compounds described herein.

In one aspect, this invention relates to a method for reducing weight, which can include administering to a subject in need of such treatment an effective amount of one or more of the compounds described herein.

In another aspect, this invention relates to a method for reducing weight gain, which can include administering to a subject in need of such treatment an effective amount of one or more of the compounds described herein.

In a further aspect, this invention relates to the use of one or more of the compounds described herein for the manufacture of a medicament for use in the prophylaxis or treatment of a GHSR receptor-related disorder.

In some embodiments, the compounds described herein can be partial antagonists or antagonists for the GHSR receptor.

Examples of GHSR receptor-related disorders can include (a) obesity and related disorders such as diabetes type II, dyslipidemia and the metabolic syndrome Prader-Willi syndrome; (b) cardiovascular diseases such as atherosclerotic vascular disease, angina pectoris, myocardial infarction and stroke; (c) intestinal inflammation that is associated with inflammatory bowel diseases (IBD) such as Crohn's disease and ulcerative colitis; (d) acromegaly; and (e) cancer, in particular breast, lung, prostate, thyroid and endocrine pituary carcinomas.

The compounds and compositions described herein can be useful for treating one or more diseases, including, for example and without limitation, obesity and related disorders such as diabetes type II, dyslipidemia and the metabolic syndrome Prader-Willi syndrome, cardiovascular diseases such as atherosclerotic vascular disease, angina pectoris, myocardial infarction and stroke, intestinal inflammation that is associated with inflammatory bowel diseases (EBD) such as Crohn's disease and ulcerative colitis, acromegaly and cancer, in particular breast, lung, prostate, thyroid and endocrine pituary carcinomas. In one aspect, the invention relates to a method for treating or preventing any one or more of the aforementioned diseases, which includes administering to a subject in need of such treatment an effective amount of any compound(s) or composition delineated herein.

Definitions

The following definitions shall apply throughout the specification and the appended claims.

Unless otherwise stated or indicated, the term “C₁₋₆-alkyl” denotes a straight or branched alkyl group having from 1 to 6 carbon atoms. Examples of said lower alkyl include methyl, ethyl, n-propyl, iso-propyl, n-butyl, iso-butyl, sec-butyl, t-butyl and straight- and branched-chain pentyl and hexyl. For parts of the range “C₁₋₆-alkyl” all subgroups thereof are contemplated such as C₁₋₅-alkyl, C₁₋₄-alkyl, C₁₋₃-alkyl, C₁₋₂-alkyl, C₂₋₆-alkyl, C₂₋₅-alkyl, C₂₋₄-alkyl, C₂₋₃-alkyl, C₃₋₆-alkyl, C₄₋₅-alkyl, etc. “C₁₋₆-alkylcarbamoyl” means a carbamoyl group substituted by a C₁₋₆-alkyl group. “C₁₋₆-alkyl ester of N-glycylcarbonyl” means that a carbonyl group is bonded the N-terminal of a C₁₋₆-alkyl ester of glycine. “C₁₋₆-alkylsulfonyl” means a sulfonyl group bonded to a C₁₋₆-alkyl group.

Unless otherwise stated or indicated, the term “C₂₋₆-alkenyl” denotes a straight or branched alkenyl group having from 2 to 6 carbon atoms. Examples of said alkenyl include vinyl, allyl, 1-butenyl, 1-pentenyl, and 1-hexenyl. For parts of the range “C₂₋₆-alkenyl” all subgroups thereof are contemplated such as C₂₋₅-alkenyl, C₂₋₄-alkenyl, C₂₋₃-alkenyl, C₃₋₆-alkenyl, C₃₋₅-alkenyl, C₃₋₄-alkenyl, C₄₋₆-alkenyl, C₄₋₅-alkenyl, etc. “C₂₋₆-alkenylcarbamoyl” means a carbamoyl group substituted by a C₂₋₆-alkenyl group.

Unless otherwise stated or indicated, the term “C₁₋₆-acyl” denotes a straight or branched acyl group having from 1 to 6 carbon atoms. Examples of said lower acyl include formyl, acetyl, propionyl, n-butyryl, 2-methylpropionyl, n-pentoyl, and n-hexoyl. For parts of the range “C₁₋₆-acyl” all subgroups thereof are contemplated such as C₁₋₅-acyl, C₁₋₄-acyl, C₁₋₃-acyl, C₁₋₂-acyl, C₂₋₆-acyl, C₂₋₅-acyl, C₂₋₄-acyl, C₂₋₃-acyl, C₃₋₆-acyl, C₄₋₅-acyl, etc.

Unless otherwise stated or indicated, the term “C₃₋₈-cycloalkyl” denotes a cyclic alkyl group having a ring size from 3 to 8 carbon atoms. Examples of said cycloalkyl include cyclopropyl, cyclobutyl, cyclopentyl, cyclohexyl, methylcyclohexyl, cycloheptyl, and cyclooctyl. For parts of the range “C₃₋₈-cycloalkyl” all subgroups thereof are contemplated such as C₃₋₇-cycloalkyl, C₃₋₆-cycloalkyl, C₃₋₅-cycloalkyl, C₃₋₄-cycloalkyl, C₄₋₈-cycloalkyl, C₄₋₇-cycloalkyl, C₄₋₆-cycloalkyl, C₄₋₅-cycloalkyl, C₅₋₇-cycloalkyl, C₆₋₇-cycloalkyl, etc. “C₃₋₈-cycloalkylcarbamoyl” means a carbamoyl group substituted by a C₃₋₈-cycloalkyl group.

Unless otherwise stated or indicated, the term “C₁₋₆ alkoxy” denotes a straight or branched alkoxy group having from 1 to 6 carbon atoms. Examples of said lower alkoxy include methoxy, ethoxy, n-propoxy, iso-propoxy, n-butoxy, iso-butoxy, sec-butoxy, t-butoxy and straight- and branched-chain pentoxy and hexoxy. For parts of the range “C₁₋₆-alkoxy” all subgroups thereof are contemplated such as C₁₋₅-alkoxy, C₁₋₄-alkoxy, C₁₋₃-alkoxy, C₁₋₂-alkoxy, C₂₋₆-alkoxy, C₂₋₅-alkoxy, C₂₋₄-alkoxy, C₂₋₃-alkoxy, C₃₋₆-alkoxy, C₄₋₅-alkoxy, etc.

Unless otherwise stated or indicated, the term “halogen” shall mean fluorine, chlorine, bromine or iodine.

Unless otherwise stated or indicated, the term “aryl” refers to a hydrocarbon ring System having at least one aromatic ring. Examples of aryls are phenyl, pentalenyl, indenyl, indanyl, isoindolinyl, chromanyl, naphthyl, fluorenyl, anthryl, phenanthryl and pyrenyl. The aryl rings may optionally be substituted with C₁₋₆-alkyl. Examples of substituted aryl groups are 2-methylphenyl and 3-methylphenyl. Likewise, aryloxy refers to an aryl group bonded to an oxygen atom.

The term “heteroaryl” means in the present description a monocyclic, bi- or tricyclic aromatic ring System (only one ring need to be aromatic) having from 5 to 14, preferably 5 to 10 ring atoms such as 5, 6, 7, 8, 9 or 10 ring atoms (mono- or bicyclic), in which one or more of the ring atoms are other than carbon, such as nitrogen, sulfur, oxygen and selenium as part of the ring System. Examples of such heteroaryl rings are pyrrole, imidazole, thiophene, furan, thiazole, isothiazole, thiadiazole, oxazole, isoxazole, oxadiazole, pyridine, pyrazine, pyrimidine, pyridazine, pyrazole, triazole, tetrazole, chroman, isochroman, quinoline, quinoxaline, isoquinoline, phthalazine, cinnoline, quinazoline, indole, isoindole, indoline (i e 2,3-dihydroindole), isoindoline (i e 1,3-dihydroisoindole), benzothiophene, benzofuran, 2,3-dihydrobenzofuran, isobenzofuran, benzodioxole, benzothiadiazole, benzotriazole, benzoxazole, 2,1,3-benzoxadiazole, benzopyrazole, 2,1,3-benzothiazole, 2,1,3-benzoselenadiazole, benzimidazole, indazole, benzodioxane, 2,3-dihydro-1,4-benzodioxine, indane, 1,2,3,4-tetrahydroquinoline, 3,4-dihydro-2H-1,4-benzoxazine, 1,5-naphthyridine, 1,8-naphthyridine, pyrido[3,2-b]thiophene, acridine, fenazine and xanthene.

The term “heterocyclic” and “heterocyclyl” in the present description is intended to include unsaturated as well as partially and fully saturated mono-, bi- and tricyclic rings having from 4 to 14, preferably 4 to 10 ring atoms having one or more heteroatoms (e.g., oxygen, sulfur, or nitrogen) as part of the ring System and the reminder being carbon, such as, for example, the heteroaryl groups mentioned above as well as the corresponding partially saturated or fully saturated heterocyclic rings. Exemplary saturated heterocyclic rings are azetidine, pyrrolidine, piperidine, piperazine, morpholine, thiomorpholine, 1,4-oxazepane, azepane, phthalimide, indoline, isoindoline, 1,2,3,4-tetrahydroquinoline, 1,2,3,4-tetrahydroisoquinoline, hexahydroazepine, 3,4-dihydro-2(1H)isoquinoline, 2,3-dihydro- 1H-indole, 1,3-dihydro-2H-isoindole, azocane, 1-oxa-4-azaspiro[4,5]dec-4-ene, decahydroisoquinoline, 1,2-dihydroquinoline, and 1,4-diazepane.

“Pharmaceutically acceptable” means being useful in preparing a pharmaceutical composition that is generally safe, non-toxic and neither biologically nor otherwise undesirable and includes being useful for veterinary use as well as human pharmaceutical use.

“Treatment” as used herein includes prophylaxis of the named disorder or condition, or amelioration or elimination of the disorder once it has been established.

“An effective amount” refers to an amount of a compound that confers a therapeutic effect on the treated subject. The therapeutic effect may be objective (i.e., measurable by some test or marker) or subjective (i.e., subject gives an indication of or feels an effect).

The term “prodrug forms” means a pharmacologically acceptable derivative, such as an ester or an amide, which derivative is biotransformed in the body to form the active drug. Reference is made to Goodman and Gilman's, The Pharmacological basis of Therapeutics, 8^{th} ed., Mc-Graw-Hill, Int. Ed. 1992, “Biotransformation of Drugs”, p. 13-15.

When two of the above-mentioned terms are used together, it is intended that the latter group is substituted by the former. For example, C₃₋₆-alkenylcarbamoyl means a carbamoyl group that is substituted by a C₃₋₆-alkenyl group. Likewise, C₁₋₆-alkylsulfonyl means a sulfonyl group that is substituted by a C₁₋₆-alkyl group.

The following abbreviations have been used:

ACN means acetonitrile,

AcOH means acetic acid,

CHO means Chinese hamster ovary,

BINAP means 2,2′-Bis(diphenylphosphino)-1,1′-binaphthyl

DCM means dichloromethane,

DEPT means distortion enhancement polarisation transfer,

DMF means dimethylformamide,

DMSO means dimethyl sulfoxide,

DPPP means 1,3-bis(diphenylphosphino)propane

EI means electron ionization

ELS means electron light scattering,

HPLC means high performance liquid chromatography,

HRMS means high resolution mass spectrometry

rt means room temperature,

RT means retention time,

TEA means triethylamine,

TFA means trifluoroacetic acid,

THF means tetrahydrofuran,

All isomeric forms possible (pure enantiomers, diastereomers, tautomers, racemic mixtures and unequal mixtures of two enantiomers) for the compounds delineated are within the scope of the invention. Such compounds can also occur as cis- or trans-, E- or Z- double bond isomer forms. All isomeric forms are contemplated.

The compounds of Formula (I) may be used as such or, where appropriate, as pharmacologically acceptable salts (acid or base addition salts) thereof. The pharmacologically acceptable addition salts mentioned above are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulfuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulfonic acid, toluenesulfonic acid, methanesulfonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

For clinical use, the compounds of the invention are formulated into pharmaceutical formulations for oral, rectal, parenteral or other mode of administration. Pharmaceutical formulations are usually prepared by mixing the active substance, or a pharmaceutically acceptable salt thereof, with conventional pharmaceutical excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like.

The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner.

In a further aspect the invention relates to methods of making compounds of any of the formulae herein comprising reacting any one or more of the compounds of the formulae delineated herein, including any processes delineated herein. The compounds of Formula (I) above may be prepared by, or in analogy with, conventional methods.

The processes described above may be carried out to give a compound of the invention in the form of a free base or as an acid addition salt. A pharmaceutically acceptable acid addition salt may be obtained by dissolving the free base in a suitable organic solvent and treating the solution with an acid, in accordance with conventional procedures for preparing acid addition salts from base compounds. Examples of addition salt forming acids are mentioned above.

The compounds of Formula (I) possess a chiral carbon atom (and can possess more than one chiral carbon atoms), and they may therefore be obtained in the form of optical isomers, e.g., as a pure enantiomer, or as a mixture of enantiomers (racemate) or as a mixture containing diastereomers. The separation of mixtures of optical isomers to obtain pure enantiomers is well known in the art and may, for example, be achieved by fractional crystallization of salts with optically active (chiral) acids or by chromatographic separation on chiral columns. The chemicals used in the synthetic routes delineated herein may include, for example, solvents, reagents, catalysts, and protecting group and deprotecting group reagens. Examples of protecting groups are t-butoxycarbonyl (Boc), benzyl and trityl (triphenylmethyl). The methods described above may also additionally include steps, either before or after the steps described specifically herein, to add or remove suitable protecting groups in order to ultimately allow synthesis of the compounds. In addition, various synthetic steps may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagentsfor Organic Synthesis, John Wiley and Sons (1995) and subsequent editions thereof.

The necessary starting materials for preparing the compounds of Formula (I) are either known or may be prepared in analogy with the preparation of known compounds. The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

The invention will now be further illustrated by the following non-limiting Examples. The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### Experimental Methods

All reagents were commercial grade and were used as received without further purification, unless otherwise specified. The chemicals were bought from Sigma-aldrich (The old brickyard, New road, Gillingham, Dorset, SP8 4XT, UK), Lancaster (Eastgate, White Lund, Morecambe, Lancashire, LA3 3DY, UK), and Acros (Bishop Meadow road, Loughborough, leicestershire, LE11 5RG, UK). Commercially available anhydrous solvents were used for reactions conducted under inert atmosphere. Reagent grade solvents were used in all other cases, unless otherwise specified

¹H nuclear magnetic resonance (NMR) and ¹³C NMR were recorded on a Bruker PMR 500 spectrometer at 500.1 MHz and 125.1 MHz, respectively, on a Bruker Advance DPX 400 spectrometer at 400.1 and 100.6 MHz, respectively or on a JEOL eclipse 270 spectrometer at 270.0 MHz and 67.5 MHz, respectively. Chemical shifts for ¹H NMR spectra are given in part per million and either tetramethylsilane (0.00 ppm) or residual solvent peaks were used as internal reference. Splitting patterns are designated as follows: s, singlet; d, doublet; t, triplet; q, quartet; p, pentet; m, multiplet; br, broad. Coupling constants are given in Hertz (Hz). Only selected data are reported. Chemical shifts for ¹³C NMR spectra are expressed in parts per million and residual solvent peaks were used as internal reference.

Electrospray mass spectrometry (MS) was obtained using an Agilent MSD mass spectrometer. Accurate mass measurements were performed on a Micromass LCT dual probe. The microwave heatings were made in a SmithCreator from Personal Chemistry.

Analytical HPLC were performed on Agilent 1100 system equipped with System A: ACE 3 (C8, 50×3.0 mm) or System B: YMC ODS-AQ, (33×3.0 mm) using the eluent system: water/0.1% TFA and CH₃CN, 1 mL/min, with a gradient time of 3 min.

Preparative HPLC was performed on a Gilson system equipped with System A: ACE 5 C8 column (50×20 mm) gradient time 5 min, system B: YMC ODS-AQ (150×30 mm) gradient time 8.5 min, system C: YMC ODS-AQ (50×20mm) gradient time 5 min or System D: ACE 5 C8 column (150×30 mm) gradient time 8.5 min using the eluent system: water/0.1% TFA and CH₃CN.

Preparative flash chromatography was performed on Merck silica gel 60 (230-400 mesh). Compounds were named using ACD/Name, version ACD/Labs 6.00 from Advanced Chemistry Development Inc.

### EXAMPLES

### Example 1

### 1′-(2-phenoxyethyl)-6-(trifluoromethyl)-2,3,4,9-tetrahydrospiro[beta-carboline-1,3′-pyrrolidine]

2-(3-chloropropyl)-1,3-dioxolane (350 μL, 2.7 mmol) was added to [4-(trifluoromethyl)phenyl]hydrazine (468 mg, 2.7 mmol) in ethanol (25 mL) and water (5 mL) and the reaction was heated at 95° C. for 1 h and the solvent was than removed in vacuo. The crude 2-[5-(trifluoromethyl)-1H-indol-3-yl]ethanamine was purified by preparative HPLC (System B). 1-(2-phenoxyethyl)pyrrolidin-3-one (148 mg, 0.72mmol) in acetic acid (1 mL) was added to the 2-[5-(trifluoromethyl)-1H-indol-3-yl]ethanamine (164.5, 0.72 mmol) and the reaction was heated at 100° C. for 1 h, diluted with methanol (2 mL) and purified by preparative HPLC (System B) to afford 6.0 mg (2%) of product.

HPLC 100%, RT: 2.023 (System A, 10-97% ACN over 3 min).

1H NMR (270 MHz, Methanol-d₃) δ ppm 2.64-2.72 (m, 2 H) 3.09-3.14 (m, 2 H) 3.37-4.88 (m, 8 H) 4.29-4.33 (m, 2 H) 6.93-6.99 (m, 3 H) 7.26-7.31 (m, 2 H) 7.40-7.53 (m, 2 H) 7.86 (s, 1 H).

HRMS (El) Calcd for C₂₃H₂₄F₃N₃O 415.1871, found 415.1881.

### Example 2

### 1′-(2-Phenoxyethyl)-6-(1H-tetrazol-5-yl)-2,3,4,9-tetrahydrospiro[beta-carboline-1,3′-Pyrrolidine]

A heck vial was charged with 1′-(2-phenoxyethyl)-2,3,4,9-tetrahydrospiro[beta-carboline-1,3′-pyrrolidine]-6-carbonitrile (50 mg, 0.134 mmol), trimethylsilyl azide (62 mg, 0.538 mmol), and dibutyltin oxide (3.3 mg, 10 mol %). Dry toluene (2 mL) was added. The vial was flushed with nitrogen, sealed with a cap and heated at 100° C. for 24 h. LC-MS indicated full conversion. The reaction mixture was transferred to a round-bottomed flask using methanol to dissolve residual material. Evaporation in vacuo gave a yellow gum that was subjected to purification on a FlashTube eluting with DCM/MeOH (5:1 v/v) to give 26 mg (46%) of a yellowish solid.

HPLC 100%, R_{T}=1.38 min (System A. 10-97% ACN over 3 min), 100%, R_{T}=1.29 min (System B. 10-90% ACN over 3 min).

¹H NMR (400 MHz, DMSO-d6) δ 2.10-2.16 (m, 1 H) 2.25-2.32 (m, 1 H) 2.76-2.85 (m, 3 H) 2.90-2.99 (m, 3 H) 3.04-3.12 (m, 2 H) 3.21 (t, J=5.8 Hz, 2 H) 4.14 (t, J=5.7 Hz, 2 H) 6.90-6.95 (m, 3 H) 7.26-7.30 (m, 2 H) 7.37 (d, J=8.5 Hz, 1 H) 7.77 (dd, J=8.4, 1.4 Hz, 1 H) 8.05 (s, 1 H) 11.00 (s, NH)

HRMS (EI) Calcd for C₂₃H₂₅N₇O: 415.2121, found 415.2113

### Example 3

### 1′-(2-phenoxyethyl)-2,3,4,9-tetrahydrospiro[beta-carboline-1,3′-pyrrolidine]-6-carbonitrile

1′-(2-phenoxyethyl)-2,3,4,9-tetrahydrospiro[beta-carboline-1,3′-pyrrolidin]-6-yl trifluoromethanesulfonate (300 mg, 0.61 mmol), NaCN (60 mg, 1.21 mmol) and tetrakis(triphenylphosphine)palladium (60 mg, 10 mol %) were suspended in dry acetonitrile in a microwave vial. The suspension was degassed under vacuum and flushed with nitrogen (3 times) to remove oxygen, and the vial was sealed with a cap. Heating with microwave irradiation in a Smith Creator for 30 min at 120° C. gave approximately 60% conversion to the product nitrile (LC-MS). The reaction mixture was poured into water (20 mL) and the aqueous phase was extracted with ethyl acetate (3×20 mL). The combined organic layers were dried over sodium sulfate, filtered and evaporated in vacuo leaving a yellow oil (311 mg). The crude oil was purified by flash column chromatography on silica gel eluting with DCM/MeOH (10:1 v/v) yielding 192 mg of a white solid (85% yield, contained 10% triphenylphosphine oxide). 70 mg of this material was subjected to flash chromatography on silica gel utilizing DCM/MeOH (20:1 v/v) as the eluent. This gave 60 mg of the pure title compound.

HPLC 96%, R_{T}=1.58 min (System A. 10-97% ACN over 3 min), 98%, R_{T}=1.48 min (System B. 10-90% ACN over 3 min).

¹H NMR (400 MHz, CDCl₃) δ 2.04-2.11 (m, 1 H) 2.27-2.35 (m, 1 H) 2.50 (d, J=8.5 Hz, 1 H) 2.60-2.76 (m, 3 H) 2.94-3.14 (m, 3 H) 3.22-3.34 (m, 3 H) 4.09-4.18 (m, 2 H) 6.93-7.00 (m, 3 H) 7.24 (d, 1 H, obscured by CDCl₃ peak) 7.28-7.34 (m, 3 H) 7.78 (s, 1H) 9.61 (s, 1 H)

HRMS (EI) Calcd for C₂₃H₂₄N₄O: 372.1950, found 372.1958

### Example 4

### 1′-(2-Phenoxyethyl)-2,3,4,9-tetrahydrospiro[beta-carboline-1,3′-pyrrolidine]-6-carboxamide

In a vial, 1′-(2-phenoxyethyl)-2,3,4,9-tetrahydrospiro[beta-carboline-1,3′-pyrrolidine]-6-carbonitrile (50 mg, 0.134 mmol) was dissolved in EtOH (2.5 mL) and 2M aq. KOH (0.5 mL) was added. The vial was sealed with a screwcap and heated to reflux. After 48 h, approx 60% conversion to the carboxamide was observed (LC-MS). A spatula of solid KOH was added to the reaction mixture, and heating continued for 5 h. The conversion was about 70%, and the reaction was stopped. All the volatiles were evaporated and the yellow oily residue was subjected to purification with a FlashTube, eluting with DCM/MeOH (10:1 v/v). The fluorescent band contained both product carboxamide and starting material (42 mg, white solid). The solid was taken up in hot DCM, and filtered. The white residual solid (18 mg) was >90% pure.

HPLC 90%, R_{T}=1.25 min (System A. 10-97% ACN over 3 min), 91%, R_{T}=1.16 min (System B. 10-90% ACN over 3 min).

¹H NMR (400 MHz, DMSO-d6) δ 1.89-1.95 (m, 1 H) 2.12-2.19 (m, 1 H) 2.57-2.60 (m, 2 H) 2.79-3.02 (m, 8 H) 4.11 (t, J=5.8Hz, 2 H) 6.89-6.95 (m, 3 H) 7.01 (br s, NH) 7.25-7.29 (m, 3 H) 7.58 (dd, J=8.5, 1.5 Hz, 1 H) 7.77 (br s, NH) 7.79 (s, 1 H) 10.79 (s, N) HRMS (EI) Calcd for C₂₃H₂₆N₄O₂: 390.2056 ,found 390.2048

### Example 5

### 1′-(2-Phenoxyethyl)-2,3,4,9-tetrahvdrospiro[beta-carboline-1,3′-pyrrolidine]-6 carboxylic acid bis(trifluoroacetate)

A microwave vial was charged with 1′-(2-phenoxyethyl)-2,3,4,9-tetrahydrospiro[beta-carboline-1,3′-pyrrolidin]-6-yl trifluoromethanesulfonate (100 mg, 0.202 mmol), BINAP (13 mg, 10 mol %), Mo(CO)₆ (53 mg, 0.202 mmol) and Herrman's catalyst (10 mg, 5 mol %). Diglyme (1 mL), ethylene glycol (1 mL), toluene (1 mL) and 4M aq. K₂CO₃ (0.18 mL, 0.72 mmol) were added. The vial was sealed with a cap and irradiated for 15 min in a Smith Creator at 150° C. The dark green reaction mixture was filtered and evaporated in vacuo. The aqueous phase was washed with chloroform (10 mL, twice). All the product resided in the aqueous phase (LC-MS). The aqueous phase (containing ethylene glycol) was subjected to preparative HPLC (System D) yielding 11 mg of pure product (di-TFA salt).

HPLC 100%, R_{T}=1.40 min (System A. 10-97% ACN over 3 min), 100%, R_{T}=1.29 min (System B. 10-90% ACN over 3 min).

¹H NMR (400 MHz, DMSO-d6, 80° C.) δ 2.34-2.40 (m, 1 H) 2.43-2.47 (m, 1 H) 2.97-2.99 (m, 2 H) 3.13-3.35 (m, 6 H, obscured by HDO peak) 3.42-3.45 (m, 2 H) 4.23 (t, J=5.5 Hz, 2 H) 6.93-6.97 (m, 3 H) 7.27-7.31 (m, 2 H) 7.47 (d, J=8.5 Hz, 1 H) 7.77 (dd, J=8.6, 1.7 Hz, 1 H) 8.15 (d, J=1.3 Hz, 1 H) 11.26 (s, NH)

HRMS (EI) Calcd for C₂₃H₂₅N₃O₃: 391.1896, found 391.1896.

### Example 6

### 6-(Morpholin4-ylcarbonyl)-1′-(2-phenoxyethyl)-2,3,4,9-tetrahydrospiro[beta-carboline-1,3′-pyrrolidine]bis(trifluoroacetate)

A microwave vial was charged with 1′-(2-phenoxyethyl)-2,3,4,9-tetrahydrospiro[beta-carboline-1,3′-pyrrolidin]-6-yl trifluoromethanesulfonate (100 mg, 0.20 mmol), BINAP (13 mg, 10 mol %), Mo(CO)₆ (53 mg, 0.20 mmol) and Herrman's catalyst (10 mg, 5 mol %). Diglyme (1 mL), toluene (1 mL), 4 M aq. K₂CO₃ (0.18 mL, 0.72 mmol), and morpholine (22 L, 0.26 mmol) were added. The vial was sealed with a cap and irradiated for 15 min in a Smith Creator at 150° C. The dark green reaction mixture was taken up in aqueous sat. sodium carbonate (10 mL), extracted with chloroform (25 mL twice), and dried over sodium sulfate. Filtration and evaporated in vacuo gave 75 mg of a green gum. The desired product was detected (LC-MS, about 10% product). The crude material was purified by preparative HPLC using 17-40% ACN (System D) yielding 4 mg of a beige gum.

HPLC 94%, R_{T}=1.40 min (System A. 10-97% ACN over 3 min), 93%, R_{T}=1.27 min (System B. 10-97% ACN over 3 min).

¹H NMR (400 MHz, MeOD-d4, contains two rotamers 70:30) δ 2.58-2.66 (m, 1 H) 2.69-2.77 (m, 1 H) 3.08-3.14 (m, 2 H) 3.41-3.73 (m, 12 H) 3.85-3.91 (m, 1 H) 4.30-4.33 (m, 2 H) 6.94-6.98 (m, 3 H) 7.26-7.30 (m, 2.7 H) 7.43-7.48 (m, 1 H) 7.64 (d, J=1.0 Hz, 0.7 H) 7.90 (dd, J=8.5, 1.8 Hz, 0.3 H) 8.27 (d, J=1.0 Hz, 0.3 H).

HRMS (El) Calcd for C₂₇H₃₂N₄O₃: 460.2474, found 460.2473

### Example 7

### N,N-Dimethyl-1′-(2-phenoxyethyl)-2,3,4,9-tetrahydrospiro[beta-carboline-1,3′-pyrrolidine]-6-carboxamide bis(trifluoroacetate)

A Heck-vial was charged with 1′-(2-phenoxyethyl)-2,3,4,9-tetrahydrospiro[beta-carboline-1,3′-pyrrolidin]-6-yl trifluoromethanesulfonate (50 mg, 0.10 mmol), BINAP (7 mg, 10 mol %) Mo(CO)₆ (53 mg, 0.20 mmol), Herrman's catalyst (5 mg, 5 mol %) and dimethyl amine HCl (35 mg, 0.43 mmol). DME (1 mL), toluene (1 mL), and 4 M aqueous K₂CO₃ (0.1 mL, 0.40 mmol)) were added. The vial was sealed with a cap and heated to 110° C. After 2 h, conversion was about 50%. Heating was continued overnight but no further conversion observed (LC-MS). The mixture was evaporated in vacuo, taken up in MeOH (1.5 mL), filtered and purified by preparative HPLC using 17-40% ACN (System D). Pure fractions were pooled and evaporated in vacuo, leaving 7 mg (17%) of a colorless gum.

HPLC 97%, R_{T}=1.37 min (System A. 10-97% ACN over 3 min), 100%, R_{T}=1.25 min (System B. 10-97% ACN over 3 min).

¹H NMR (400 MHz, MeOD-d4, contains two rotamers 60:40) δ 2.67-2.74 (m, 1 H) 2.78-2.85 (m, 1 H) 3.06-3.15 (m, 6 H) 3.57-3.69 (m, 6 H) 3.70-3.76 (m, 1 H) 3.80-3.86 (m, 1 H) 4.05-4.10 (m, 1 H) 4.35 (t, J=4.9 Hz, 2 H) 6.95-6.99 (m, 3 H) 7.26-7.31 (m, 2.6 H) 7.44-7.48 (m, 1 H) 7.63 (d, J=1.0 Hz, 0.6 H) 7.91 (dd, J=8.7, 1.6 Hz, 0.4 H) 8.30 (d, J=1.0 Hz, 0.4 H)

HRMS (El) Calcd for C₂₅H₃₀N₄O₂: 418.2369, found 418.2356.

### Example 8

### N,N-Dimethyl-1′-(2-phenoxyethyl)-2,3,4,9-tetrahydrospiro[beta-carboline-1,3′-pyrrolidine]-6-carboxamide bis(trifluoroacetate)

A Heck-vial was charged with 1′-(2-phenoxyethyl)-2,3,4,9-tetrahydrospiro[beta-carboline-1,3′-pyrrolidin]-6-yl trifluoromethanesulfonate, BINAP, Mo(CO)₆, Herrman's catalyst and ethylamine HCl. Diglyme, toluene, and aqueous K₂CO₃ were added. The vial was sealed with a cap and heated at 110° C. for 3 h. Conversion was about 60-70% (LC-MS). The volatiles were evaporated and methanol was added (total volume 1.7 mL) and filtered. The crude material was purified by preparative HPLC using 17-38% ACN (System D) and pure fractions were evaporated in vacuo yielding 13 mg of a colorless gum. Closer analysis revealed contamination with the benzoic acid derivative (ca 40%). A second purification using 32-55% ACN (5 mM NH₄OAc) gave the pure ethyl amide (3 mg, mono-acetate)

HPLC 99%, R_{T}=1.40 min (System A. 10-97% ACN over 3 min), 100%, R_{T}=1.18 min (System B. 10-97% ACN over 3 min).

¹H NMR (400 MHz, MeOD-d4) δ 1.23 (t, J=7.3 Hz, 3 H) 1.93 (s, 3 H) 2.21-2.27 (m, 1 H) 2.36-2.44 (m, 1 H) 2.90 (t, J=5.9 Hz, 2 H) 2.98-3.13 (m, 6 H) 3.42 (q, J=7.1 Hz, 2 H) 4.20 (t, J=5.3 Hz, 2 H) 6.90-6.96 (m, 3 H) 7.22-7.28 (m, 2 H) 7.35 (d, J=8.5 Hz, 1 H) 7.61 (dd, J=8.8, 1.8 Hz, 1 H) 7.99 (d, J=1.0 Hz, 1 H)

HRMS (EI) Calcd for C₂₅H₃₀N₄O₂: 418.2369, found 418.2366

### Example 9

### 6-(Ethylthio)-1′-(2-phenoxyethyl)-2,3,4,9-tetrahydrospiro[b-carboline-1,3′-pyrrolidine]bis(trifluoroacetate)

AlCl₃ (24 mg, 0.18 mmol) was suspended in DCM. Under a nitrogen atmosphere was added 1′-(2-phenoxyethyl)-2,3,4,9-tetrahydrospiro[beta-carboline-1,3′-pyrrolidin]-6-ol (50 mg, 0.14 mmol) yielding a yellow suspension. The mixture was cooled on an ice-bath to 0° C. and ethanethiol (200 μL, excess) was added. The suspension was allowed to warm to room temperature and stirred for 1 h. No reaction had occurred and an additional amount of 200 μL ethanethiol was added. Stirring continued for 3 days (no conversion) and further AlCl₃ (40 mg, 0.30 mmol) was added giving a deep yellow suspension. After 18 h, the reddish solution was analyzed (LC-MS). The reaction mixture contained product (10%), dehydroxylated material (50%) and starting material (40%). The thioether product was isolated by preparative HPLC (System D) using 28-51% ACN. Evaporation of the pure fractions gave 9 mg (10%) of the title compound as a yellow gum.

HPLC 100%, Rₜ=1.88 min (System A. 10-97% ACN over 3 min), 100%, R_{T}=1.68 min (System B. 10-97% ACN over 3 min).

¹H NMR (400 MHz, MeOD-d4) δ 1.21 (t, J=7.4 Hz, 1.5 H) 1.28 (t, J=7.3 Hz, 1.5 H) 2.61-2.68 (m, 1 H) 2.72-2.78 (m, 1 H) 2.87 (q, J=7.4 Hz, 1 H) 2.95 (q, J=7.4 Hz, 1 H) 3.07 (t, J=6.2 Hz, 1 H) 3.47-3.53 (m, 4 H) 3.59-3.65 (m, 3 H) 3.72-3.78 (m, 1 H) 3.96 (d, J=12.6 Hz, 1 H) 4.33 (t, J=4.9 Hz, 2 H) 4.49 (s, 2 H) 6.94-7.00 (m, 3 H) 7.04-7.07 (m, 0.5 H) 7.11-7.15 (m, 0.5 H) 7.22-7.31 (m, 3 H) 7.34-7.36 (m, 0.5 H) 7.58 (d, J=1.0 Hz, 0.5 H)

MS (ESI+) m/z 408 (M+H).

### Example 10

### 1-[1′-(2-phenoxyethyl)-2,3,4,9-tetrahydrospiro[b-carboline-1,3′-pyrrolidin]-6-yl]ethanone trifluoroacetate

1′-(2-phenoxyethyl)-2,3,4,9-tetrahydrospiro[β-carboline-1,3′-pyrrolidin]-6-yl trifluoromethanesulfonate (50.0 mg, 0.10 mmol), butyl vinyl ether (12.6 mg, 0.12 mmol), palladium acetate (2.3 mg, 0.01 mmol), DPPP (4.3 mg, 0.01) and triethylamine (14.1 μL, 0.10 mmol) were all suspended in DMF (90 μL)and H₂O (15 μL). The vial was sealed with a cap and the reaction mixture was de-gassed and filled with N₂ and then heated at 100° C. for 300 s using microwaves. The reaction mixture was filtrated and purified by directly by preparative HPLC (System B) 20-42 % ACN. The fractions containing product were combined to give a yellow oil (2.9 mg, 7%).

HPLC 100%, R_{T}=1.516 min (System A. 10-97 % ACN), 100%, R_{T}=1.344 min (System B. 10-97 % ACN).

¹H NMR (400 MHz, MeOD) δ ppm 2.58-2.62 (m, 1 H) 2.65 (s, 3 H) 2.69-2.77 (m, 1 H) 3.14 (t, J=5.8 Hz, 2 H) 3.40-3.73 (m, 7 H) 3.88 (d, J=12.3 Hz, 1 H) 4.32 (t, J=5.0 Hz, 2 H) 6.94-6.98 (m, J=8.3, 8.3 Hz, 3 H) 7.26-7.30 (m, 2 H) 7.45 (d, J=8.7 Hz, 1 H) 7.89 (dd, J=8.7, 1.7 Hz, 1 H) 8.27 (d, J=1.5 Hz, 1 H)

HRMS (EI) Calcd for C₂₄H₂₇N₃O₂: 389.2103, found 389.2088

Alternative Synthesis:

5-acetyl-3-(2-aminoethyl)-1H-indole-2-carboxylic acid prepared by the procedure of Shavel, von Strandtmann and Cohen [JACS 84: 881 (1962)] (90 mg, 0.35 mmol) and 1-(2-phenoxyethyl)pyrrolidin-3-one (110 mg, 0.52 mmol) were dissolved in acetic acid (4mL) and heated to 150° C. for 1800 s under microwave irradiation. The reaction mixture was evaporated and toluene (10 mL) was added and evaporated to remove remaining acetic acid. Further toluene was added and decanted off. The residue was dissolved in hot ethanol (50 mL) and filtered to remove insoluble material. After evaporation, the crude product was purified by preparative HPLC (System B) 10-35% acetonitrile 0.1% TFA to afford the product as a gum (38 mg, 29%)

HPLC 91% R_{T}=1.56 (System A. 10-97% ACN over 3 min) 91% R_{T}=1.41 (System B. 10-90% ACN over 3 min)

¹H NMR (400 MHz, MeOD) δ ppm 2.67 (s, 3 H) 2.75 (dt, J=15.06, 7.53 Hz, 1 H) 2.80-2.90 (m, 1 H) 3.18 (t, J=6.15 Hz, 2 H) 3.57 (q, 1 H) 3.63-3.68 (m, 2 H) 3.68-3.74 (m, 3 H) 3.81 (d, J=13.30 Hz, 1 H) 3.88 (ddd, J=10.98, 7.28, 7.09 Hz, 1 H) 4.12 (d, J=13.05 Hz, 1 H) 4.35-4.41 (m, 2 H) 6.98-7.03 (m, J=8.78 Hz, 3 H) 7.31 (dd, J=8.91, 7.40 Hz, 2 H) 7.50 (d, J=9.29 Hz, 1 H) 7.93 (dd, J=8.53, 1.76 Hz, 1 H) 8.30 (s, 1 H)

Chiral separation of 1-[1′-(2-phenoxyethyl)-2,3,4,9-tetrahydrospiro[b-carboline-1,3′-pyrrolidin]-6-yl]ethanone into the two individual stereoisomers were performed using a chiral preparative column (Chirobiotic V, 5 U, 250×21.2 mm). Mobile phase: 20 mM NH40ac, pH 2.51/MeOH (69/31).

After evaporation, the products from the hplc separation were dissolved in little DCM and HCl in methanol (0.2 mL) was added. Evaporation gave an off white solid.

Optical rotation of the more active enantiomer was measured in ethanol. Specific rotation [a]_{D}=−33.3°

### Example 11

### Methyl(2E)-3-[1′-(2-phenoxyethyl)-2,3,4,9-tetrahydrospiro[β-carboline-1,3′-pyrrolidinl-6-yl]acrylate trifluoroacetate

1′-(2-phenoxyethyl)-2,3,4,9-tetrahydrospiro[β-carboline-1,3′-pyrrolidin]-6-yl trifluoromethanesulfonate (50.0 mg, 0.10 mmol), methyl acrylate (13.6 μL, 0.15 mmol), palladium acetate (2.3 mg, 0.01 mmol), DPPP (4.3 mg, 0.01) and triethylamine (14.1 μL, 0.10 mmol) were all suspended in DMF (500 μL). The vial was sealed with a cap and the reaction mixture was de-gassed and filled with N₂ and then heated at 100° C. for 600s using microwaves. The solvent was removed under reduced pressure and the remaining brown oil was diluted in MeOH, filtered and purified directly by preparative HPLC (System B) 22-44% ACN. The fractions containing product were combined to give a yellow oil (2.5 mg, 6%).

HPLC 99%, R_{T}=1.735 min (System A. 10-97% ACN), 100%, R_{T}=1.565 min (System B. 10-97 % ACN).

¹H NMR (400 MHz, MeOD) δ ppm 2.56-2.63 (m, 1 H) 2.67-2.74 (m, 1 H) 3.09-3.13 (m, 1 H) 3.32-3.48 (m, 4 H) 3.61-3.68 (m, 3 H) 3.78 (s, 3 H) 3.83 (d, J=12.8 Hz, 1 H) 4.31 (m, 2 H) 6.46 (d, J=15.9 Hz, 1 H) 6.94-6.98 (m, 3 H) 7.26-7.30 (m, 2 H) 7.41 (d, J=32 8.6 Hz, 1 H) 7.51 (dd, 1 H) 7.76 (d, J=1.5 Hz, 1 H) 7.79 (d, J=16.0 Hz, 1 H)

HRMS (EI) Calcd for C₂₆H₂₉N₃O₃: 431.2209, found 431.2200

### Example 12

### (2E)-3-[1′-(2-phenoxyethyl)-2,3,4,9-tetrahydrospiro[b-carboline-1,3′-pyrrolidin]-6-yl]acrylamide trifluoroacetate

1′-(2-phenoxyethyl)-2,3,4,9-tetrahydrospiro[β-carboline- 1,3′-pyrrolidin]-6-yl trifluoromethanesulfonate (50.0 mg, 0,10 mmol), acryl amide (10.8 mg, 0.15 mmol), palladium acetate (2.3 mg, 0.01 mmol), DPPP (4.3 mg, 0.01) and triethylamine (14.1 μL, 0.10 mmol) were all suspended in DMF (500 μL). The vial was sealed with a cap and the reaction mixture was de-gassed and filled with N₂ and then heated at 100° C. for 600 s using microwaves. The solvent was removed under reduced pressure and the remaining brown oil was diluted in MeOH, filtered and purified directly by preparative HPLC (System B) 14-35% ACN. The fractions containing product were combined to give a yellow oil (3.9 mg, 6%).

HPLC 99%, R_{T}=1.354 min (System A. 10-97% ACN), 100%, R_{T}=1.211 min (System B. 10-97% ACN).

¹H NMR (400 MHz, MeOD) δ ppm 2.6-2.70 (m, 1 H) 2.73-2.81 (m, 1 H) 3.06-3.13 (m, 2 H) 3.47-3.81 (m, 7 H) 3.96-4.02 (dd, J=12.0, 10.0 Hz, 1 H) 4.33 (t, J=4.4 Hz, 1 H) 5.7 (m, 1 H) 6.6 (d, J=15.7 Hz, 1 H) 7.0 (m, J=8.5, 7.6 Hz, 3 H) 7.5 (m, 1 H) 7.7 (m. 1 H).

HRMS (EI) Calcd for C₂₅H₂₈N₄O₂: 416.2212, found 416.2205

### Example 13

### Ethyl 1′-(2-phenoxyethyl)-2,3,4,9-tetrahydrospiro[β-carboline-1,3′-pyrrolidine]-6-carboxylate

1′-(2-phenoxyethyl)-2,3,4,9-tetrahydrospiro[β-carboline-1,3′-pyrrolidin]-6-yl trifluoromethanesulfonate (155.0 mg, 0.30 mmol), palladium acetate (10.5 mg, 0.05 mmol), DPPP (19.4 mg, 0.05) and triethylamine (50 μL, 0.30 mmol) were all dissolved in ethanol (15 mL). The clear yellow solution was heated and vigourosly stirred at 80° C. in an atmosphere of carbon monoxide (balloon) over the weekend. The darkened mixture was filtered through a small pad of silica, the solvent was evaporated and the residue was chromatographed on a column of silica initially with CHCl₃ 100% followed by 95/5/0.2 CHCl₃/MeOH/aq conc NH₃ to give 0.11 g (85%) of the target compound as a brown oil.

¹H NMR (400 MHz, CHLOROFORM-D) d ppm 1.41 (t, J=7.15 Hz, 3 H) 2.03-2.11 (m, 1 H) 2.28-2.37 (m, 1 H) 2.52 (d, J=8.66 Hz, 1 H) 2.61-2.69 (m, 1 H) 2.72-2.77 (m, 2 H) 2.93-3.06 (m, 2 H) 3.08-3.16 (m, 1 H) 3.21 -3.33 (m, 3 H) 4.07-4.19 (m, 2 H) 4.38 (q, J=7.15 Hz, 2 H) 6.90-7.01 (m, 3 H) 7.23 (dd, J=8.53, 0.50 Hz, 1 H) 7.27-7.33 (m, 2 H) 7.83 (dd, J=8.53, 1.63 Hz, 1 H) 8.19-8.25 (m, 1 H) 9.44 (s, 1 H). ¹³C NMR (CDCl₃) δ 14.44, 22.35, 39.21, 41.51, 53.02, 54.07, 59.78, 60.41, 65.89, 66.13, 107.60, 110.45, 114.47, 120.92, 121.09, 122.79, 126.70, 129.59, 138.09, 141.45, 158.56, 167.93.

HPLC 96%, R_{T}=1.74 min (System A. 10-97% ACN), 94%, R_{T}=1.56 min (System B. 10-93% ACN).

HRMS (EI) Calcd for C₂₅H₂₉N₃O₃: 419.2209, found 419.2203

### Example 14

### Methyl 1′-(2-phenoxyethyl)-2,3,4,9-tetrahydrospiro[β-carboline-1,3′-pyrrolidine]-6-carboxylate

To a solution of 1′-(2-phenoxyethyl)-2,3,4,9-tetrahydrospiro[β-carboline-1,3′-pyrrolidin]-6-yl trifluoromethanesulfonate (2.0 g, 4.0 mmol) in MeOH (15 mL) were added dppp (0.25 g 0.6 mmol), Pd(OAc)₂ (0.136 g, 0.6 mmol), TEA (0.45 g, 4.4 mmol) and the clear yellow solution was vigourously stirred at 63° C. under an CO-atmosphere (balloon) for three days. The reaction was not complete so another portion of dppp, Pd(OAc)₂ and TEA were added to the solution and the reaction was continued for a further three days. The reaction mixture was filtered through a small pad of silica, the solvent was removed at reduced pressure and the residue was purified by preparative HPLC (System D). The solvent from the pure fractions were removed at reduced pressure and the residue was taken up between CHCl₃ and ice cold 0.5M NaOH to give 1.00 g (61%) the free base of the target compound as a light yellow crisp.

¹H NMR (400 MHz, CHLOROFORM-D) δ ppm 2.04-2.13 (m, 1 H) 2.29-2.39 (m, 1 H) 2.55 (d, J=8.78 Hz, 1 H) 2.63-2.77 (m, 3 H) 2.94-3.17 (m, 3 H) 3.21 -3.36 (m, 3 H) 3.91 (s, 3 H) 4.08-4.20 (m, 2 H) 6.89-7.02 (m, 3 H) 7.20-7.34 (m, 3 H) 7.83 (dd, J=8.53, 1.76 Hz, 1 H) 8.19-8.25 (m, 1 H) 9.45 (s, 1 H).

¹³C NMR (CDCl₃) δ 22.33, 39.12, 41.68, 51.69, 52.97, 53.93, 59.77, 65.74, 65.82, 107.52, 110.57, 114.49, 120.97, 121.01, 121.12, 122.84, 126.80, 129.59, 138.18, 141.68, 158.54, 168.31.

HPLC 100%, Rₜ=1.6 min (System A. 10-97% ACN), 100%, R_{T}=1.40 min (System B. 10-97% ACN).

LC-MS; MH+=406.

### Example 15

### 6-(Methylthio)-1′-(2-phenoxyethyl)-2,3,4,9-tetrahydrospiro[beta-carboline-1,3′-pyrrolidine]

4-Thiomethylphenylhydrazine hydrochloride (1.06 g, 5.56 mmol) and 3-chloropropyldioxolane (0.84 g, 5.56 mmol) were refluxed in EtOH:H₂O (10 mL, 5:1 v/v) for 4 h. The dark red reaction mixture was allowed to cool to room temperature and the volatiles were removed in vacuo, giving 1.89 g of a dark brown syrup that was directly used in the next step.

This crude {2-[5-(methylthio)-1H-indol-3-yl]ethyl}amine hydrochloride was dissolved in acetic acid (30 mL) and treated with 1-(2-phenoxyethyl)pyrrolidin-3-one (1.60 g, 7.80 mmol). The reaction mixture was heated in a STEM block at 95° C. for 3 h. After all the tryptamine starting material had been consumed (LC-MS), the dark brown reaction mixture was allowed to cool to room temperature and the volatiles were removed in vacuo. The residual dark oil (>5 g) was taken up in DCM (200 mL) and washed with 2 N aq. NH₄OH (200 mL). The aqueous layer was extracted with DCM (150 mL) and the combined DCM layers dried over Na₂SO₄. Filtration and evaporation in vacuo gave 2.63 g of brown oil. Purification by flash chromatography on silica gel, eluting with DCM/MeOH (20:1 v/v) gave 0.51 g of a yellow oil (starting ketone), and 0.31 g of brown oil (product). The product was further purified by flash chromatography on silica gel, eluting with chloroform (sat. NH₃). This gave 280 mg of a light brown solid (13%)

HPLC 96%, R_{T}=1.75 min (System A. 10-97% ACN over 3 min), 96%, R_{T}=1.54 min (System B. 10-97% ACN over 3 min).

¹H NMR (400 MHz, CDCl₃) δ 2.02-2.08 (m, 1 H) 2.26-2.33 (m, 1 H) 2.45-2.49 (m, 1 H) 2.49 (s, 3 H) 2.58-2.75 (m, 3 H) 2.92-3.04 (m, 2 H) 3.07-3.13 (m 1 H) 3.20-3.29 (m, 3 H) 4.08-4.17 (m, 2 H) 6.93-6.98 (m, 3 H) 7.14-7.20 (m, 2 H) 7.28-7.33 (m, 2 H) 7.49 (m, 1 H) 9.12 (s, NH).

HRMS (ED) Calcd for C₂₃H₂₇N₃OS: 393.1875, found 393.1862.

### Example 16

### 6-(Methylsulfonyl)-1′-(2-phenoxyethyl)-2,3,4,9-tetrahvdrospiro[beta-carboline-1,3′-pyrrolidine]

6-(methylthio)-1′-(2-phenoxyethyl)-2,3,4,9-tetrahydrospiro[beta-carboline-1,3′-pyrrolidine] (270 mg, 0.69 mmol) was dissolved in acetonitrile (dry, 5 mL) and DMF (dry, 2.5 mL). Sodium iodide (0.62 g, 4.14 mmol) and titanium(IV) chloride (220 μL, 2.01 mmol) were added under N₂-atm. The resulting brown suspension was stirred for 10 min at room temperature, mildly heated for 5 min and then quenched with aq. KOH (diluted, 20 mL). Brine (50 mL) was added and the mixture was extracted with chloroform (100 mL). The chloroform layer was dried over Na₂SO₄, filtered and evaporated in vacuo leaving 280 mg of a brown oil. Purification by flash chromatography on silica gel, eluting with chloroform (sat. ammonia) gave 64 mg of product (87-91% pure). A second flash column on silica gel, eluting with DCM/MeOH (10:1 v/v) gave an ivory solid (37 mg, 13%).

HPLC 96%, Rₜ=1.43 min (System A. 10-97% ACN over 3 min), 98%, Rₜ=1.24 min (System B. 10-97% ACN over 3 min).

¹H NMR (400 MHz, CDCl₃) δ 2.05-2.12 (m, 1 H) 2.28-2.35 (m, 1 H) 2.50 (d, J=8.8 Hz, 1 H) 2.60-2.67 (m, 1 H) 2.72-2.75 (m, 2 H) 2.95-3.15 (m, 3 H) 3.05 (s, 3 H) 3.23 (d, J=8.5 Hz, 1 H) 3.26-3.34 (m, 2 H) 4.09-4.18 (m, 2 H) 6.94-7.00 (m, 3 H) 7.29-7.34 (m, 3 H) 7.63 (dd, J=8.5, 2.0 Hz, 1 H) 8.09 (d, J=1.8 Hz, 1 H) 9.66 (s, NH).

HRMS (EI) Calcd for C₂₃H₂₇N₃O₃S: 425.1773, found 425.1756.

### Example 17

### N-[1′-(2-phenoxyethyl)-2,3,4,9-tetrahydrospiro[beta-carboline-1,3′-pyrrolidin]-6-yl]methanesulfonamide trifluoroacetate)

3-(2-aminoethyl)-5-[(methylsulfonyl)amino]-1H-indole-2-carboxylic acid prepared by the procedure of Larsen and Gould (U.S. Pat. No. 3,472,870) (100 mg, 0.35 mmol) and 1-(2-phenoxyethyl)pyrrolidin-3-one (70 mg, 0.35 mmol) were dissolved in acetic acid (4mL) and heated to 140° C. for 900 s under microwave irradiation. The reaction mixture was evaporated and toluene (10 mL) was added and evaporated to remove remaining acetic acid. The residue was dissolved in CH₂Cl₂:methanol 9:1 and passed though a pad of silica gel. After evaporation, the crude product was purified by preparative HPLC (System B) 10-40% acetonitrile 0.1% TFA to afford the product as a white solid (31 mg, 21%)

HPLC 100% R_{T}=1.27 (System A. 10-97% ACN over 3 min) 99% R_{T}=1.44 (System B. 10-90% ACN over 3 min)

¹H NMR (500 MHz, MeOD) δ ppm 2.70 (ddd, J=14.91, 7.38, 7.22 Hz, 1 H) 2.77- 2.83 (m, 1 H) 2.79 (s, 3 H) 3.00 (t, J=5.97 Hz, 2 H) 3.57 - 3.63 (m, J=6.75, 6.75, 6.75, 6.75 Hz, 2 H) 3.63-3.68 (m, 2 H) 3.68-3.75 (m, 1 H) 3.83-3.88 (m, 2 H) 3.89 (s, 2 H) 4.14 (d, J=13.50 Hz, 1 H) 4.31 (t, J=4.87 Hz, 2 H) 6.87-6.92 (m, 3 H) 7.06 (dd, J=8.48, 1.88 Hz, 1 H) 7.20 (t, J=8.01 Hz, 2 H) 7.31 (d, J=8.79 Hz, 1 H) 7.37 (s, 1 H)

HRMS (EI) calcd for C₂₃H₂₈N₄O₃S: 440.1882, found 440.1875

### Example 18

### 1-[2-acetyl-1′-(2-phenoxyethyl)-2,3,4,9-tetrahydrospiro[beta-carboline-1,3′-pyrrolidin]-6-yl]ethanone trifluoroacetate

1-[1′-(2-phenoxyethyl)-2,3,4,9-tetrahydrospiro[beta-carboline- 1,3′-pyrrolidin]-6-yl]ethanone (19 mg, 0.05 mmol) in acetonitrile (0.5 mL) was treated with acetic anhydride (5 μL, 0.1 mmol) and the mixture heated to 70° C. for 36 hours. The crude reaction mixture was purified directly by preparative HPLC (System B) 10-35% acetonitrile 0.1% TFA to afford the product as a gum (5 mg, 21 %).

HPLC 99% R_{T}=1.92 (System A. 10-97% ACN over 3 min) 99% R_{T}=1.70 (System B. 10-90% ACN over 3 min)

¹H NMR (400 MHz, MeOD) δ ppm 2.36 (s, 3 H) 2.68 (s, 3 H) 2.74 (dd, J=13.43, 9.16 Hz, 1 H) 2.82-2.94 (m, 1 H) 2.97 (s, 2 H) 3.64-3.70 (m, 2 H) 3.74 (d, J=12.55 Hz, 1 H) 3.81-3.90 (m, 2 H) 4.04-4.15 (m, 1 H) 4.21-4.32 (m, 1 H) 4.34-4.45 (m, 3 H) 6.98-7.09 (m, 3 H) 7.33 (t, J=7.53 Hz, 2 H) 7.49 (d, J=8.53 Hz, 1 H) 7.91 (d, J=8.53 Hz, 1 H) 8.28 (s, 1 H)

### Example 19

### O-[1′-(2-Phenoxyethyl)-2,3,4,9-tetrahvdrospiro[b-carboline-1,3′-pyrrolidinl-6-yl]dimethylcarbamate

Under a nitrogen atmosphere, a Heck-vial was charged with NaH (14 mg, 0.58 mmol) and dry THF (2 mL). 1′-(2-phenoxyethyl)-2,3,4,9-tetrahydrospiro[beta-carboline-1,3′-pyrrolidin]-6-ol (150 mg, 0.41 mmol) was added causing H₂-evolution. After 5 min, the dark brown solution was treated with the dimethylaminocarbamoyl chloride (42 μL, 0.46 mmol). The vial was heated to 50° C. in a STEM block and stirred for 1 h. The volatiles were removed in vacuo leaving 268 mg of a brown oil. The crude was purified by flash chromatogragphy on silica gel eluting with DCM/MeOH (20:1 v/v) affording a white solid (115 mg, 64%).

HPLC 100%, R_{T}=1.60 min (System A. 10-97% ACN over 3 min), 100%, R_{T}=1.40 min (System B. 10-97% ACN over 3 min).

¹NMR (400 MHz, CDCl₃) δ 2.02-2.08 (m, 1 H) 2.27-2.34 (m, 1 H) 2.50 (d, J=8.8 Hz, 1 H) 2.60-2.68 (m, 3 H) 2.93-3.09 (m, 3 H) 3.01 (s, 3 H) 3.11 (s, 3H) 3.19-3.30 (m, 3 H) 4.09-4.18 (m, 2 H) 6.84 (dd, J=8.5, 2.5 Hz, 1 H) 6.93-6.97 (m, 3 H) 7.16 (s, 1 H) 7.18 (d, J=6.5 Hz, 1 H) 7.27-7.31 (m, 2 H) 9.01 (br s, NH).

HRMS (ED) Calcd for C₂₅H₃₀N₄O₃: 434.2318, found 434.2311.

### Example 20

### 1′-(2-Phenoxyethyl)-2,3,4,9-tetrahydrospiro[beta-carboline-1,3′-pyrrolidinl-6-yl trifluoromethanesulfonate

1′-(2-phenoxyethyl)-2,3,4,9-tetrahydrospiro[beta-carboline- 1,3′-pyrrolidin]-6-ol (10.0 g, 27.5 mmol) and N,N-bis-(trifluoromethanesulfonyl)aniline (10.8 g, 30.3 mmol) were dissolved in DCM (300 mL). Methanol (4 mL) followed by triethylamine (4 mL, 28.7 mmol) was added at room temperature and the starting materials dissolved. After 18 h, the reaction was not complete (LC-MS), so more triflating agent was added (5 g) and stirring continued for 3 h. The volatiles were removed in vacuo, and the crude product (32.5 gram) was purified by flash column chromatography on silica gel eluting with DCM/methanol (40:1 v/v). The product was isolated as light brown foam (10.52 g, 77%). A fraction preceding the product gave a brown crude oil containing the N-methylated product (due to the methanol, via methylsulfonate, 3.17 g). This impure fraction was taken up in DCM (200 mL) and washed with 2M ammonium hydroxide (150 mL, twice) to remove N-trifluoromethanesulfonylaniline. The DCM-layer was dried over Na₂SO₄ and evaporated in vacuo leaving 1.11 g of brown oil. Purification with flash chromatography on silica gel eluting with chloroform/methanol (25:1 v/v) gave 0.82 g (6%) of brown oil.

HPLC 98%, Rₜ=2.01 min (System A. 10-97% ACN over 3 min), 99%, R_{T}=1.89 min (System B. 10-90% ACN over 3 min).

¹H NMR (400 MHz, CDCl₃) δ 2.04-2.10 (m, 1 H) 2.27-2.35 (m, 1 H) 2.50 (d, J=8.5 Hz, 1 H) 2.61-2.75 (m, 3 H) 2.94-3.14 (m, 3 H) 3.23-3.33 (m, 3 H) 4.08-4.17 (m, 2 H) 6.93-7.00 (m, 4 H) 7.21 (d, J=8.8 Hz, 1 H) 7.28-7.33 (m, 3 H) 9.50 (s, 1 H)

MS (ESI+) m/z 496 (M+H)

### Example 21

### 2-Methyl-1′-(2-Phenoxyethyl)-2,3,4,9-tetrahvdrospiro[beta-carboline-1,3′-pyrrolidin]-6-yl trifluoromethanesulfonate

HPLC 98%, R_{T}=2.18 min (System A. 10-97% ACN over 3 min), 98%, R_{T}=1.95 min (System B. 10-97% ACN over 3 min).

¹H NMR (400 MHz, CDCl₃) δ 2.12-2.20 (m, 1 H) 2.24-2.31 (m, 1 H) 2.41 (s, 3 H) 2.51-2.60 (m, 2 H) 2.68 (d, J=8.3 Hz, 1 H) 2.90-3.20 (m, 6 H) 3.28-3.34 (m, 1 H) 4.16 (t, J=5.3 Hz, 2 H) 6.97-7.03 (m, 4 H) 7.19 (d, J=8.8 Hz, 1 H) 7.32-7.37 (m, 3 H) 9.73 (s, 1 H)

HRMS (EI) Calcd for C₂₄H₂₆F₃N₃O₄S: 509.1596, found 509.1586.

### Example 22

### O-[1′-(2-Phenoxyethyl)-2,3,4,9-tetrahydrospiro[b-carboline-1,3′-pyrrolidin]-6-yl] dimethylthiocarbamate

Under an atmosphere of nitrogen, a Heck-vial was charged with NaH (18.6 mg, 0.78 mmol) and dry THF (3 mL). 1′-(2-phenoxyethyl)-2,3,4,9-tetrahydrospiro[beta-carboline-1,3′-pyrrolidin]-6-ol (200 mg, 0.55 mmol) was added causing H₂-evolution. After 5 min, the dark brown solution was treated with the dimethylaminothiocarbamoyl chloride (75 mg, 0.61 mmol). The vial was heated to 60° C. in a STEM block and stirred for 1 h. The reaction mixture was poured into water (30 mL) and extracted with DCM (3×15 mL). Combined organic layers were dried (Na₂SO₄) and evaporated in vacuo leaving 268 mg of brown oil. The crude was purified by flash chromatography on silica gel eluting with DCM/MeOH (10:1 v/v) affording an ivory white solid (203 mg, 82%).

HPLC 100%, R_{T}=1.71 min (System A. 10-97% ACN over 3 min), 100%, R_{T}=1.52 min (System B. 10-97% ACN over 3 min).

¹H NMR (400 MHz, MeOD-d4) δ 2.08-2.15 (m, 1 H) 2.28-2.35 (m, 1 H) 2.70 (t, J=5.7 Hz, 2 H) 2.84-3.13 (m, 8 H) 3.35 (s, 3 H) 3.42 (s, 3 H) 4.15-4.20 (m, 2 H) 6.72 (dd, J=8.8, 2.3 Hz, 1 H) 6.90-6.96 (m, 3 H) 7.00 (d, J=2.3 Hz, 1 H) 7.22-7.29 (m, 3 H).

HRMS (ED) Calcd for C₂₅H₃₀N₄O₂S: 450.2089, found 450.2089.

### Example 23

### 2-(2-amino-2-oxoethyl)-1′-(2-phenoxyethyl)-2,3,4,9-tetrahydrospiro[beta-carboline-1,3′-pyrrolidin]-6-yl trifluoromethanesulfonate trifluoroacetate

1′-(2-Phenoxyethyl)-2,3,4,9-tetrahydrospiro[beta-carboline-1,3′-pyrrolidin]-6-yl trifluoromethanesulfonate (58 mg, 0.12 mmol), 2-bromoacetamide (16mg, 0.12mmol) and potassium carbonate (16 mg, 0.12 mmol) were combined with DMF (2mL) and heated to 100° C. for 2 hours, cooled to room temperature, filtered and the solvent removed. The crude product was purified directly by preparative HPLC (System D, 37-61% ACN) to give the desired product (9 mg, 12%).

HPLC 100%, R_{T}=2.18 min (System A. 10-97% ACN over 3 min), 100%, R_{T}=1.98 min (System B. 10-97% ACN over 3 min).

¹H NMR (400 MHz, MeOD) δ ppm 2.54-3.01 (m, 4 H) 3.32-3.37 (m, 1 H) 3.39 -3.85 (m, 7 H) 3.94-4.18 (m, 2 H) 4.32-4.44 (m, 2 H) 6.95-7.05 (m, 3 H) 7.10 (dd, J=8.79, 2.55 Hz, 1 H) 7.26-7.36 (m, 2 H) 7.37-7.49 (m, 2 H)

HRMS (EI) Calcd for C₂₅H₂₇F₃N₄O₅S: 552.1654, found 552.1647.

### Example 24

### 1′-[2-(4-Cyanophenoxy)ethyl]-2,3,4,9-tetrahydrospiro[beta-carboline-1,3′-pyrrolidin]-6-yl dimethylcarbamate trifluoroacetate (single enantiomer)

4-{2-[6-hydroxy-2,3,4,9-tetrahydro-1′H-spiro[P-carboline- 1,3′-pyrrolidin]-1′-yl]ethoxy}benzonitrile (a single but unidentified enantiomer) (12 mg, 0.03 mmol), dimethylcarbamoyl chloride (2.8 μL, 0.03 mmol), potassium carbonate (12 mg) were combined in dry ACN (0.5 mL) and stirred at 50° C. for 3 hours. The solution was filtered and the crude product was purified directly by preparative HPLC (System D, 20-50% ACN) to give the desired product (3.5 mg, 25%).

HPLC 98%, R_{T}=2.1.57 min (System A. 10-97% ACN over 3 min), 100%, R_{T}=1.38 min (System B. 10-97% ACN over 3 min).

¹H NMR (400 MHz, MeOD) δ ppm 2.5 (m, 1 H) 2.6 (m, 1 H) 3.0 (s, 3 H) 3.1 (m, 3 H) 3.1 (s, 3 H) 3.2 (m, 3 H) 3.5 (m, 1 H) 3.6 (m, 3 H) 4.3 (t, J=5.0 Hz, 2 H) 6.9 (dd, J=8.7, 2.3 Hz, 1 H) 7.1 (m, 2 H) 7.2 (d, J=1.9 Hz, 1 H) 7.3 (d, J=8.8 Hz, 1 H) 7.7 (m, 2 H)

HRMS (ED) Calcd for C₂₆H₂₉N₅O₃: 459.2270, found 459.2259

### Example 25

### Ethyl ({[6-acetyl-1′-(2-phenoxyethyl)-4,9-dihydrospiro[beta-carboline-1,3′-pyrrolidin]-2(3H)-yl]carbonyl}amino)acetate trifluoroacetate

Ethyl isocyanatoacetate (5 μL, 0.045 mmol) was added to 1-[1′-(2-phenoxyethyl)-2,3,4,9-tetrahydrospiro[beta-carboline-1,3′-pyrrolidin]-6-yl]ethanone (19 mg, 0.041 mmol) in acetonitrile (1 mL) and stirred at RT for 2 h. The solution was filtered and the crude product was purified directly by preparative HPLC (System C, 30-60% ACN) to give the desired product (11 mg, 43%).

HPLC 99%, R_{T}=2.052 min (System A. 10-97% ACN over 3 min), 99%, R_{T}=1.222 min (System B. 30-80% ACN over 3 min).

¹H NMR (400 MHz, MeOD) δ ppm 1.15 (t, J=7.03 Hz, 3 H) 2.52-2.60 (m, 3 H) 2.58 -2.69 (m, 1 H) 2.69-2.82 (m, 1 H) 2.81-2.91 (m, 2 H) 3.49-3.82 (m, 6 H) 3.86 (d, J=4.27 Hz, 2 H) 4.03 (q, 2 H) 4.04-4.13 (m, 1 H) 4.13-4.22 (m, J=12.80 Hz, 1 H) 4.24-4.32 (m, 2 H) 6.82-7.03 (m, J=8.03, 8.03 Hz, 2 H) 7.16-7.29 (m, 2 H) 7.37 (d, J=8.78 Hz, 2 H) 7.80 (dd, J=8.66, 1.63 Hz, 1 H) 8.16 (d, J=1.51 Hz, 1 H)

### Example 26-27

### 2-[(2R)-2,3-dihydroxypropyl]-1′-(2-phenoxyethyl)-2,3,4,9-tetrahydrospiro[β-carboline-1,3′-pyrrolidine]-6-carboxylic acid;

### 2-[(2S)-2,3-dihydroxypropyl]-1′-(2-phenoxyethyl)-2,3,4,9-tetrahydrospiro[β-carboline-1,3′-pyrrolidine]-6-carboxylic acid

1′-(2-phenoxyethyl)-2,3,4,9-tetrahydrospiro[β-carboline-1,3′-pyrrolidin]-6-yl trifluoromethanesulfonate (0.235 g, 0.5 mmol) and methyl (2R)-oxirane-2-carboxylate (200 mg, 2 mmol) were heated together to 100° C. under microwave irradiation for 1 hour to give crude intermediate Methyl (2R)-2-hydroxy-3-[1′-(2-phenoxyethyl)-6-{[(trifluoromethyl)sulfonyl]oxy}-4,9-dihydrospiro[β-carboline-1,3′-pyrrolidin]-2(3H)-yl]propanoate.

This crude material was dissolved in THF (1 ml) and added to a suspension of lithium borohydride (0.22g, 10 mmol) in THF (10 ml) cooled in an icebath. The mixture was stirred at 0° C. for 1 hour. Saturated aqueous NH₄Cl (4 ml) and water (50 ml) were added and the solution extracted with EtOAc. The organic phase was separated, dried (Na₂SO₄) and evaporated to give a yellow foam. The compound was found to have epimerized under the reduction conditions. 2-(2,3-dihydroxypropyl)-1′-(2-phenoxyethyl)-2,3,4,9-tetrahydrospiro[b-carboline-1,3′-pyrrolidin]-6-yl trifluoromethanesulfonate. Yield 173mg (61% over 2 steps)

The above triflate was dissolved in ethanol (5 ml). In a flask flushed with N₂ were added the triflate solution, Pd(OAc)2, dppp & TEA. The nitrogen was replaced by carbon monoxide and the mixture was stirred under CO (g) at 80° C. overnight.

The mixture was filtered and the solvents removed in vacuum. The crude product ethyl 2-(2,3-dihydroxypropyl)-1′-(2-phenoxyethyl)-2,3,4,9-tetrahydrospiro[b-carboline- 1,3′-pyrrolidine]-6-carboxylate was purified using flash chromatography, gradient 2-10% MeOH in EtOAc.

The above ester was hydrolysed by dissolving in methanol (20 mL) and water (10 mL) and dividing between 5 microwave reaction tubes. Lithium hydroxide (50 mg) was added to each tube and they were heated to 100° C. for 10 minutes with microwave irradiation.

The mixtures were combined, filtered and made slightly acidic with acetic acid. After evaporation, the crude products (two diastereoisomers) were separated by preparative hplc 23% MeCN isocratic 77% 0.1% TFA YMC column.

The two compounds (peak 1 and 2 from the hplc) had a specific optical rotation [a]_{D} (methanol:water 2:1) of −4.2° and 4.2° repectively.

### Biological Methods

Chinese hamster ovary cells (CHO), cell line (ES-410-F) purchased from Euroscreen, stably expressing the human GHSR seeded in 96 well plates are pre-loaded with Fluo-4AM fluorescent dye for 60 min before addition of test compounds (5 μM for primary screen). Fluorescent intensity is recorded using a Fluorometric imaging plate reader (FLIPR 98R 96-well format, Molecular Devices) and inhibition of the peak response evoked by ghrelin (EC₇₀ concentration) is calculated.

Potency (IC₅₀) determinations are performed utilizing the same functional assay as described for primary screening, applying the compounds in the concentration range of 170 pM to 10 μM or 340 pM to 20 μM.

The calculation of the functional Kᵢ values for the inhibitors was performed by use of Activity Base. The Kᵢ value is calculated from IC₅₀ using the Cheng Prushoff equation (with reversible inhibition that follows the Michaelis-Menten equation): Kᵢ=IC₅₀ (1+[S]/Kₘ) (Cheng, Y. C.; Prushoff, W. H. (1973) Biochem. Pharmacol. 22: 3099-3108). The compounds of formula (I) exhibit Kᵢ values for human GHSR in the range from 1 nM to 1 μM. See for example the following table:

| Compound | GHSR-human Kᵢ |
|---|---|
| A | 19 nM |
| B | 111 nM |
| C | 38 nM |

What is claimed is:

1. 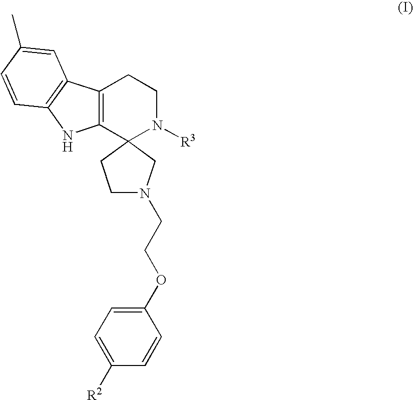
wherein R¹ is a member selected from the group of cyano, SR⁴, SO₂R⁴, N(R⁴)₂, CO₂R⁴, COR⁴, CON(R⁴)₂, O(CO)N(R⁴)₂, OS0₂R⁴, O(CS)N(R⁴)₂, N(R⁴)(CO)N(R⁴)₂, N(R⁴)(CO)R⁴, N(R⁴)(SO₂)R⁴, CH═CHCOOR⁴, CH═CHCON(R⁴)₂, (CH₂)ₙCOOR⁴, (CH₂)ₙCON(R⁴)₂, halo or perhaloalkyl, or a heterocycle wherein each R⁴ is independently selected from hydrogen, aryl, heteroaryl, (C₁₋₆)alkyl, (C₂₋₆)alkenyl, (C₂₋₆)alkynyl-1,1(C₂₋₈)heteroalkyl, halo(C₁₋₆)alkyl, or per halo(C₁₋₆)alkyl; or two R⁴ groups attached to the same nitrogen atom are combined to form a five- to eight-membered heterocyclic ring; and wherein R⁴ is optionally substituted with from one to three substituents independently selected from the group consisting of halogen, hydroxyl, alkoxy, or oxo;
n is 1 to 4;
R² is hydrogen or cyano; and
R³ is selected from hydrogen, an alkyl ester of N-glycylcarbonyl, C₁₋₆-alkyl ester of N-glycylacetyl, carbamoyl-C₁₋₆-alkyl, N-C₁₋₆-alkylcarbamoyl-C₁₋₆-alkyl, N,N-C₁₋₆-dialkylcarbamoyl-C₁₋₆-alkyl, N,N-C₁₋₆-dialkylcarbamoylamino-C₁₋₆-alkyl, C₁₋₆-alkoxy-C₂₋₆-acylamino-C₁₋₆-alkyl, 3-amino-1,2-dioxocyclobut-3-ene-4-ylamino-C₁₋₆-alkyl, 3-C₁₋₆-alkoxy-1,2-dioxocyclobut-3-ene-4-ylamino-C₁₋₆-alkyl, cyano-C₁₋₆-alkyl, C₁₋₆-alkoxyhydroxyalkyl, carboxy-C₁₋₆-alkyl, C₁₋₆-alkoxycarbonyl-C₁₋₆-alkyl, C₁₋₆-alkoxy-C₁₋₆-alkyl, C₁₋₆-alkoxy-C₁₋₆-alkoxy-C₁₋₆-alkyl, aryl-C₁₋₆-alkylamino-C₂₋₆-acyl, C₁₋₆-alkoxycarbonyl-C₁₋₆-alkylamino-C₂₋₆acyl, carboxy-C₁₋₆-alkylamino-C₂₋₆-acyl, C₂₋₆-acyl-C₂₋₆-acyl, aryloxy-C₁₋₆-alkyl, C₁₋₆-alkylsulfonylamino-C₁₋₆-alkyl, C₁₋₆-alkoxycarbonyl-C₂₋₆-acyl, C₁₋₆-alkoxy-C₂₋₆-acyl, C₁₋₆-alkylthio-C₂₋₆-acyl, di-C₁₋₆-alkylamino-C₂₋₆-acyl, heteroarylcarbamoyl, C₁₋₆-alkoxycarbonyl, heteroaryl-C₂₋₆-acyl, C₁₋₆-alkylsulfonyl-C₂₋₆-acyl, heterocyclyl-C₂₋₆-acyl, C₁₋₆-alkoxy-C₁₋₆-alkylamino-C₂₋₆-acyl, carboxy-C₂₋₆-acyl, amino-C₂₋₆-acyl, C₁₋₆-alkylamino-C₂₋₆-acyl, carbamoyl-C₁₋₆-alkylamino-C₂₋₆-acyl, heterocyclyl-C₁₋₆-alkyl, heteroaryl-C₁₋₆-alkyl, carbamoylamino-C₁₋₆-alkyl, hydroxy-C₂₋₆-acylcarbamoyl, C₁₋₆-alkylcarbamoyl-C₁₋₆-alkylamino-C₁₋₆-alkyl, C₁₋₆-alkoxycarbonyl-C₁₋₆-alkylamino-C₁₋₆-alkyl, amino-C₂₋₆-acylamino-C₂₋₆-acyl, C₁₋₆-alkoxy-C₂₋₆-acylamino-C₂₋₆-acyl, amino-C₂₋₆-acylamino-C₁₋₆alkyl, amino, C₂₋₆-acylamino-C₁₋₆-alkyl, heterocyclylcarbonylamino-C₁₋₆-alkyl, C₂₋₆-acylamino-C₁₋₆-alkyl, amino-C₂₋₆-acylamino-C₂₋₆-acyl, C₂₋₆-acylamino-C₂₋₆-acyl, hydroxy-C₁₋₆-alkylamino-C₂₋₆acyl, C₁₋₆-alkoxycarbonyl-C₁₋₆-alkyl, amino-C₁₋₆-alkyl, carboxy-C₁₋₆-alkyl, 2-(3-hydroxy-1,2-dioxocyclobut-3-ene-4-yl)amino-C₁₋₆-alkyl, heteroarylcarbonylamino-C₁₋₆-alkyl, carboxyamino-C₁₋₆-alkyl, N,N-di-C₁₋₆-alkylamino-C₂₋₆-acylamino-C₁₋₆-alkyl, dihydroxy-C₁₋₆-alkyl, C₂₋₆-acylcarbonyl, C₁₋₆-alkoxybenzyl, wherein the aryl group is optionally substituted by one or more of C₁₋₆-alkoxy, the heteroaryl group is optionally substituted by one or more of C₁₋₆-alkyl and the heterocyclyl is optionally substituted by one or more of oxo;
and pharmaceutically acceptable salts, hydrates, solvates, geometrical isomers, tautomers, optical isomers, N-oxides and prodrug forms thereof.

2. A compound according to claim 1, wherein R³ is selected from acetyl, allyl, alkylcarbamoyl, aminoacetyl, 2-(3-amino-1,2-dioxocyclobut-3-ene-4-ylamino)ethyl, 3-amino-3-methyl-n-butyryl, benzylaminoacetyl, n-butylcarbamoyl, carbamoylmethyl, carbamoylmethylaminoacetyl, 3-carbamoyl-n-propyl, carbethoxy, carbethoxyacetyl, 4-carbethoxy-n-butyl, carbethoxymethyl, 3-carbethoxy-n-propyl, carbomethoxyacetyl, 4-carbomethoxy-n-butyryl, 4-carboxy-n-butyl, 3-carboxy-n-propionyl, 3-carboxy-n-propyl, 3-cyano-n-propyl, cyclohexylcarbamoyl, N,N-diethylcarbamoylmethyl, diisopropylaminoacetyl, 3,4-dimethoxybenzylaminoacetyl, dimethylaminoacetyl, 2-(N,N-dimethylcarbamoylamino)ethyl, 3,5-dimethylisoxazol-4-ylcarbamoyl, 1,4-dioxo-n-pentyl, 2-(3-ethoxy-1,2-dioxocyclobut-3-ene-4-ylamino)ethyl, ethylcarbamoyl, 4-ethylcarbamoyl-n-butyl, 3-ethylcarbamoyl-n-propyl, ethyl ester of N-glycylacetyl, ethyl ester of N-glycylcarbonyl, N-ethyl-N-methylcarbamoyl, ethylthioacetyl, N-glycylacetyl, N-glycylcarbonyl, hydrogen, hydroxyacetyl, 2-hydroxyisobutyl, 2-hydroxyethyl, 2-hydroxy-3-methoxy-n-propyl, 2-hydroxy-n-propyl, 1-imidazolylacetyl, methoxyacetyl, 2-(methoxyacetylamino)ethyl, 2-(2-methoxyethoxy)ethyl, 2-methoxyethylaminoacetyl, 3-methoxy-n-propyl, methyl, methylaminoacetyl, methylsulfonyl, methylsulfonylacetyl, 2-methylsulfonylaminoethyl, 4-morpholinylacetyl, 2-(4-morpholinyl)ethyl, 3-oxo-1-piperazinylacetyl, 2-phenoxyethyl, 1-piperazinylacetyl, 2-pyridylmethyl, 2-thienylcarbamoyl, 2-carbamoylaminoethyl, hydroxyacetylcarbamoyl, 2-(N-methylcarbamoylmethylamino)ethyl, 2-carbomethoxymetylaminoethyl, 2-amino-2-methylpropionamidoacetyl, methoxyacetylaminoacetyl, 2-(2-amino-2-methylpropionamido)ethyl, 2-aminoacetylaminoethyl, 2-(4-morpholinylcarbonylamino)ethyl, 2-acetylaminoethyl, aminoacetylaminoacetyl, acetylaminoacetyl, 2-hydroxyethylaminoacetyl, carbomethoxymethyl, 2-aminoethyl, carboxymethyl, 2-(3-hydroxy-1,2-dioxocyclobut-3-ene-4-yl)aminoethyl, 2-(2-furylcarbonylamino)ethyl, 2-(5-isoxazolylcarbonylamino)ethyl, 2-carboxyaminoethyl, 2-(2-morpholinylcarbonylamino)ethyl, 2-N,N-dimethylaminoacetylaminoethyl, 4-phenoxy-n-butyl, 2,3-dihydroxy-n-propyl, acetylcarbonyl, and 4-methoxybenzyl.

3. A compound according to claim 1, wherein R³ is hydrogen, acetyl, aminocarbonylmethyl or methyl.

4. A compound according to claim 1, wherein R¹ is trifluoromethyl, 1H-tetrazol-5-yl, cyano, aminocarbonyl, carboxy, morpholin-4-ylcarbonyl, dimethylaminocarbonyl, ethylaminocarbonyl, ethylthio, acetyl, methoxycarbonylethenyl, aminocarbonylethenyl, ethoxycarbonyl, methoxycarbonyl, methylthio, methylsulfonyl, methylsulfonamido, dimethylaminocarbonyloxy, trifluoromethanesulfonyloxy, or dimethylamino(thiocarbonyl)oxy.

5. A compound according to claim 1, wherein R² is H.

6. A compound according to claim 1, which is selected from
1′-(2-phenoxyethyl)-6-(trifluoromethyl)-2,3,4,9-tetrahydrospiro[beta-carboline-1,3′-pyrrolidine:
1′-(2-phenoxyethyl)-6-(1H-tetrazol-5-yl)-2,3,4,9-tetrahydrospiro[beta-carboline-1,3′-pyrrolidine];
1′-(2-phenoxyethyl)-2,3,4,9-tetrahydrospiro[beta-carboline-1,3′-pyrrolidine]-6-carbonitrile;
1′-(2-phenoxyethyl)-2,3,4,9-tetrahydrospiro[beta-carboline-1,3′-pyrrolidine]-6-carboxamide;
1′-(2-phenoxyethyl)-2,3,4,9-tetrahydrospiro[beta-carboline-1,3′-pyrrolidine]-6-carboxylic acid;
6-(morpholin-4-ylcarbonyl)-1′-(2-phenoxyethyl)-2,3,4,9-tetrahydrospiro[beta-carboline-1,3′-pyrrolidine];
N,N-dimethyl-1′-(2-phenoxyethyl)-2,3,4,9-tetrahydrospiro[beta-carboline-1,3′-pyrrolidine]-6-carboxamide;
N,N-dimethyl-1′-(2-phenoxyethyl)-2,3,4,9-tetrahydrospiro[beta-carboline-1,3′-pyrrolidine]-6-carboxamide;
6-(ethylthio)-1′-(2-phenoxyethyl)-2,3,4,9-tetrahydrospiro[beta-carboline-1,3′-pyrrolidine];
1-[1′-(2-phenoxyethyl)-2,3,4,9-tetrahydrospiro [b-carboline-1,3′-pyrrolidin]-6-yl]ethanone;
methyl (2E)-3-[1′-(2-phenoxyethyl)-2,3,4,9-tetrahydrospiro[β-carboline-1,3′-pyrrolidin]-6-yl]acrylate;
(2E)-3-[1′-(2-phenoxyethyl)-2,3,4,9-tetrahydrospiro[beta-carboline-1,3′-pyrrolidin]-6-yl]acrylamide;
ethyl 1′-(2-phenoxyethyl)-2,3,4,9-tetrahydrospiro[β-carboline-1,3′-pyrrolidine]-6-carboxylate;
methyl 1′-(2-phenoxyethyl)-2,3,4,9-tetrahydrospiro[β-carboline-1,3′-pyrrolidine]-6-carboxylate;
6-(methylthio)-1′-(2-phenoxyethyl)-2,3,4,9-tetrahydrospiro[beta-carboline-1,3′-pyrrolidine];
6-(methylsulfonyl)-1′-(2-phenoxyethyl)-2,3,4,9-tetrahydrospiro[beta-carboline-1,3′-pyrrolidine];
N-[1′-(2-phenoxyethyl)-2,3,4,9-tetrahydrospiro[beta-carboline-1,3′-pyrrolidin]-6-yl]methanesulfonamide;
1-[2-acetyl-1′-(2-phenoxyethyl)-2,3,4,9-tetrahydrospiro[beta-carboline-1,3′-pyrrolidin]-6-yl]ethanone;
1-[2-acetyl-1′-(2-phenoxyethyl)-2,3,4,9-tetrahydrospiro[beta-carboline-1,3′-pyrrolidin]-6-yl]ethanone;
1′-(2-phenoxyethyl)-2,3,4,9-tetrahydrospiro[beta-carboline-1,3′-pyrrolidin]-6-yl trifluoromethanesulfonate;
2-methyl-1′-(2-Phenoxyethyl)-2,3,4,9-tetrahydrospiro[beta-carboline-1,3′-pyrrolidin]-6-yl trifluoromethanesulfonate;
O-[1′-(2-phenoxyethyl)-2,3,4,9-tetrahydrospiro[beta-carboline-1,3′-pyrrolidin]-6-yl]dimethylthiocarbamate;
2-(2-amino-2-oxoethyl)-1′-(2-phenoxyethyl)-2,3,4,9-tetrahydrospiro[beta-carboline-1,3′-pyrrolidin]-6-yl trifluoromethanesulfonate;
1′-[2-(4-cyanophenoxy)ethyl]-2,3,4,9-tetrahydrospiro[beta-carboline-1,3′-pyrrolidin]-6-yl dimethylcarbamate;
ethyl ({[6-acetyl-1′-(2-phenoxyethyl)-4,9-dihydrospiro[beta-carboline-1,3′-pyrrolidin]-2(3H)-yl]carbonyl}amino)acetate
2-[(2R)-2,3-dihydroxypropyl]-1′-(2-phenoxyethyl)-2,3,4,9-tetrahydrospiro[β-carboline-1,3′-pyrrolidine]-6-carboxylic acid; or
2-[(2S)-2,3-dihydroxypropyl]-1′-(2-phenoxyethyl)-2,3,4,9-tetrahydrospiro[β-carboline-1,3′-pyrrolidine]-6-carboxylic acid.

7. A compound according to claim 1, wherein the compound has one chiral carbon atom.

8. The compound of claim 7, wherein the compound is the (S)-enantiomer and is substantially free of the (R)-enantiomer.

9. The compound of claim 7, wherein the compound is the (R)-enantiomer and is substantially free of the (S)-enantiomer.

10. The compound of claim 1, wherein the compound has two chiral carbon atoms.

11. The compound of claim 10, wherein R³ is 2,3-dihydroxy-n-propyl.

12. A process for the preparation of a compound according to claim 1, which comprises:
(a) performing a carboxy mediated Pictet-Spengler reaction, or
(b) performing a palladium catalysed substitution at the 6 position of the beta-carboline ring system.

13. A pharmaceutical formulation comprising a compound according to claim 1, as active ingredient, in combination with a pharmaceutically acceptable diluent or carrier.

14. A method for the prophylaxis or treatment of a GHSR receptor-related disorder, which comprises administering to a subject in need of such treatment an effective amount of a compound according to claim 1.

15. The method according to claim 14, wherein the disorder is selected from (a) obesity and related disorders such as diabetes type II, dyslipidemia and the metabolic syndrome Prader-Willi syndrome; (b) cardiovascular diseases such as atherosclerotic vascular disease, angina pectoris, myocardial infarction and stroke; (c) intestinal inflammation that is associated with inflammatory bowel diseases (IBD) such as Crohn's disease and ulcerative colitis; (d) acromegaly; and (e) cancer, in particular breast, lung, prostate, thyroid and endocrine pituary carcinomas.

16. A method for modulating GHSR receptor activity, which comprises administering to a subject in need of such treatment an effective amount of a compound according to claim 1.

17. A method for suppressing food intake, which comprises administering to a subject in need of such treatment an effective amount of a compound according to claim 1.

18. A method for suppressing appetite, which comprises administering to a subject in need of such treatment an effective amount of a compound according to claim 1.

19. A method for reducing weight, which comprises administering to a subject in need of such treatment an effective amount of a compound according to claim 1.

20. A method for reducing weight gain, which comprises administering to a subject in need of such treatment an effective amount of a compound according to claim 1.
